(19) United States
(12) Patent Application Publication
(10) Pub. No.: US 2026/0294808 A1
(43) Pub. Date: Oct. 1, 2026

(54) BIOMIMETIC PROTEOLIPID NANOVESICLES FOR DELIVERY OF NUCLEIC ACIDS

(71) Applicant: THE METHODIST HOSPITAL, Houston, TX (US)

(72) Inventors: Manuela SUSHNITHA, Houston, TX (US); Francesca TARABALLI, Houston, TX (US); Assaf Yosef ZINGER, Houston, TX (US); Chiara MANCINO, Houston, TX (US)

(21) Appl. No.: 19/489,439

(22) PCT Filed: May 31, 2024
(86) PCT No.: PCT/US2024/031899

§ 371 Date: Dec. 2, 2025

Related U.S. Application Data
(60) Provisional application No. 63/505,913, filed on Jun. 2, 2023.

(51) Int. Cl. A61K 9/1271; A61K 31/7105; A61P 35/00; C12N 15/113; C12N 15/88

(52) U.S. Cl. CPC A61K 9/1271; A61K 31/7105; A61P 35/00; C12N 15/113; C12N 15/88; C12N 2310/14; C12N 2320/32

(57) ABSTRACT

Disclosed herein is a biomimetic proteolipid nanovesicle, comprising: an ionizable or cationic lipid; a phosphocholine-based phospholipid; a cholesterol; a leukocyte membrane protein; and an siRNA encapsulated by the biomimetic proteolipid nanovesicle; wherein the biomimetic proteolipid nanovesicle has a lipid-to-protein ratio of from about 1:65 to about 1:85 by weight. Further disclosed herein is a biomimetic proteolipid nanovesicle, comprising: an ionizable or cationic lipid; a phosphocholine-based phospholipid; a cholesterol; a leukocyte membrane protein; and an mRNA encapsulated by the biomimetic proteolipid nanovesicle; wherein the biomimetic proteolipid nanovesicle has a lipid-to-protein ratio of from about 1:80 to about 1:100 by weight.

### CROSS REFERENCE TO RELATED APPLICATIONS

This application claims the benefit of priority to U.S. Provisional Application No. 63/505,913, filed Jun. 2, 2023, which is incorporated by reference herein in its entirety.

### GOVERNMENT SUPPORT CLAUSE

This invention was made with government support under Grant Nos. 1R56-CA213859 and 5F31-CA232705 awarded by the National Institutes of Health. The Government has certain rights in the invention.

### BACKGROUND

Nanoparticles (NP) represent a broad range of drug delivery vehicles that offer the ability to target diseased sites while minimizing off-target effects. However, the complex biological milieu encountered by NP upon entry into the bloodstream poses significant biological barriers that thwart their ability to deliver their payload to the target tissue. For example, systemic administration of NP exposes them to rapid uptake and clearance by components of the mononuclear phagocyte system (MPS). As a result, these NP do not reach the target site and, thereby, do not exert their therapeutic effects. Previous efforts to overcome these challenges have included the incorporation of polyethylene glycol (PEG) to improve circulation times and conjugation of targeting moieties, such an antibodies and peptides⁵, to facilitate preferential accumulation to disease sites. Over time, increasing evidence highlighted the limitations of these strategies, such as the immune response to repeated injections of PEG and the high variability in conjugation densities of targeting moieties on NP surface.

Biomimetic NP represent an emerging class of NP that aim to address the current challenges faced by the field of nanomedicine through biomimicry of native cells. Work in this field encompasses a broad range of NP, ranging from those mimicking red blood cells to immune cells to even cancer cells. Use of these biomimetic approaches has shown how traditionally used NP platforms can now harness the features of native cells to achieve specific function while maintaining the superior delivery capabilities of a synthetic NP. Examples of this include red blood cell membrane-coated polymeric NP that achieve longer circulation times for toxin removal in the blood and chemotherapy-loaded NP cloaked with cancer cell membranes for homotypic targeting of tumor cells.

The syntheses of biomimetic NP have taken on two primary forms: top-down and bottom-up approaches. Isolation of whole cell membranes which are then applied in toto onto a synthetic NP core is an example of a top-down approach where the extracted component maintains the full biological complexity of the source. In contrast, bottom-up approaches utilize incorporation of ligands or other components as the building blocks to integrate into the final NP, such as the integration of membrane proteins into synthetic NP. While top-down approaches serve as a bridge between synthetic NP and source cells, bottom-up approaches offer more control in the tuning of the final NP formulation. Regardless of the synthesis approach utilized, maintenance of key NP physicochemical and biological characteristics, both during and after the synthesis process, is a crucial component in the engineering of these platforms. Achievement of specific functionality using these complex biomimetic NP warrants the careful and rational tuning of parameters associated with the synthesis process. Parameters such as the ratio of NP to extracted cell membrane, temperature used during the synthesis steps and post-synthesis purification process are examples of factors that must be carefully considered. The engineering of these design criteria has significant effects not only on the physicochemical properties of the NP, but also their biomimetic behavior under biological conditions.

Leukocyte-based biomimetic NP for targeting inflamed tissues (i.e., Leukosomes) were previously reported by our group. Leukosomes have demonstrated the ability to home to sites of inflammation and preferentially adhere to inflamed endothelia. Previously, the feasibility of synthesizing these NP was demonstrated using two synthesis methods—thin layer evaporation and a microfluidic-based approach. Upon synthesis, characterization of the NP verified their physiochemical properties while their biological functions were demonstrated in a local inflammation model. As inflamed endothelia are a common feature in a large number of disease conditions (e.g., tumor, sepsis, traumatic brain injury, atherosclerosis, etc.), this NP platform provides a very powerful tool for effective targeting and therapeutic cargo delivery. Furthermore, the tunability of this targeting is important for the tailoring of these NP to a specific disease condition.

Building off this foundational work, this work aimed to demonstrate the tunability of this system within the context of delivery of nucleic acids while retaining leukosome's tropism towards inflammation. In particular, this work focused on the engineering of the synthesis parameters by establishing key design criteria. These design criteria included thresholds on size and PDI, conservation of key leukocyte proteins, maintenance of the lipid bilayer structure and NP stability. Recognizing the need for ease of scalability and translational strategies for NP synthesis, this work used a microfluidic-based approach for synthesis of the NP in this study.

As the integration of proteins dictates the biological behavior of leukosomes, here this behavior was modulated by analyzing the protein:lipid (P:L) mass ratio utilized in the synthesis process. Therefore, it was hypothesized that an increase in the protein content on the NP is directly correlated to their biomimetic targeting function in vitro and in vivo.

To this end, the effects of varying the P:L ratio of the leukosomes were assessed while using a microfluidic-based, bottom-up NP synthesis process. Preservation and stability of key physiochemical (e.g., size, zeta potential, NP concentration and morphology) and biomimetic (e.g., protein integration and presence of key leukocyte biological markers) parameters were first evaluated. Then, to assess the short and long-term stability of these biomimetic NP, the changes in each of the aforementioned parameters was assessed over the duration of 21 or 28 days. From here, the different formulations were tested for in vitro targeting to inflamed endothelial cells, which are the most relevant cell population implicated in the innate targeting of leukocytes to sites of inflammation.²⁷ Furthermore, preferential accumulation to sites of inflammation within the disease context was studied using murine lipopolysaccharide-induced local inflammation (LLI), triple negative breast cancer (TNBC), and posttraumatic osteoarthritis (PTOA) in vivo models.

An improved understanding of how tuning this biomimetic NP's targeting capabilities is vital for therapeutic applications of this platform. Enhancing this behavior by increasing the P:L ratio while using native leukocyte membrane proteins encompasses a simple but powerful approach. This information can yield a reproducible, potent biomimetic NP formulation that can specifically target the site of inflammation while reducing off-target effects on healthy tissues.

### SUMMARY

In an aspect, provided is a biomimetic proteolipid nanovesicle, including: an ionizable or cationic lipid; a phosphocholine-based phospholipid; a cholesterol; a leukocyte membrane protein; and an siRNA encapsulated by the biomimetic proteolipid nanovesicle; wherein the biomimetic proteolipid nanovesicle can have a lipid-to-protein ratio of from about 1:65 to about 1:85 by weight.

In another aspect, provided is a biomimetic proteolipid nanovesicle, including: an ionizable or cationic lipid; a phosphocholine-based phospholipid; a cholesterol; a leukocyte membrane protein; and an mRNA encapsulated by the biomimetic proteolipid nanovesicle; wherein the biomimetic proteolipid nanovesicle can have a lipid-to-protein ratio of from about 1:80 to about 1:100 by weight. in yet another aspect, provided is a method of making any of the disclosed biomimetic proteolipid nanovesicles, the method including: a) dissolving a phosphocholine-based phospholipid, an ionizable or cationic lipid, and a cholesterol in an organic solvent to produce an organic lipid solution; b) dissolving a leukocyte membrane protein and an agent in water to produce an aqueous protein solution; and c) loading the organic lipid solution into an organic phase inlet of a microfluidic mixer, and loading the aqueous protein solution into an aqueous phase inlet of said microfluidic mixer; and d) adjusting flow rates of each inlet stream and a flow ratio between each inlet stream to produce the biomimetic proteolipid nanovesicles having a specified lipid-to-protein ratio by weight therefrom.

In yet still another aspect, provided is a method for the delivery of an agent into a cell, the method including introducing into the cell any of the disclosed biomimetic proteolipid nanovesicles.

In yet still another aspect, provided is a method of treating a disease or disorder in a subject in need thereof, the method including administering to the subject any of the disclosed biomimetic proteolipid nanovesicles.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

FIG. 1A, FIG. 1B, FIG. 1C, and FIG. 1D depict physicochemical properties of NPs synthesized with 3 lipid backbones. Size (FIG. 1A), polydispersity index (PDI) (FIG. 1B), zeta potential (FIG. 1C), and siRNA encapsulation efficiency (FIG. 1D) characterization of liposomes synthesized from 3 different lipid backbones revealed maintenance of size less than 200 nm and PDI less than 0.2 among all 3 formulations, while DOTAP NPs exhibited positive surface charge and lowest siRNA encapsulation efficiency. n=3-6. Results are shown as means±SEM. One-way ANOVA followed by Tukey's multiple comparison test was used to determine statistical significance. P value≤0.05 among means was considered as statistically significant.

FIG. 2A and FIG. 2B depict cytotoxicity of DAP and DLin-MC3 liposomes on 4T1 cells. Cell viability analysis following treatment with DAP (FIG. 2A) and DLin-MC3 (FIG. 2B) liposomes for up to 72 h indicated minimal decreases in cell viability at concentrations less than 500 uM in both formulations. n=4. Results are shown as means±SEM.

FIG. 3A, FIG. 3B, FIG. 3C, FIG. 3D, and FIG. 3E depict siRNA knockdown efficiency of DAP and DLin-MC3 liposomes on TNBC cells. Knockdown efficiency of STAT3 mRNA expression following treatment with DAP (FIG. 3A) and DLin-MC3 (FIG. 3B) liposomes revealed the latter induced almost 80% knockdown. FIG. 3C shows further evaluation of DLin-MC3 knockdown efficiency using heparinase siRNA-loaded liposomes corroborated the 80% knockdown efficiency of this formulation. FIG. 3D shows reduction of GFP-protein expression in MDA-MB-231-GFP cells by Lipofectamine and DLin-MC3 indicated similar levels of protein reduction by both systems for up to 72 h. FIG. 3E shows quantification of the green signal demonstrated the reduction in both the Lipo and Lipofectamine treated groups. n=3. Results are shown as means±SEM. One-way ANOVA followed by Dunnett's multiple comparison test was used to determine statistical significance. P value≤0.05 among means was considered as statistically significant.

FIG. 4A, FIG. 4B, FIG. 4C, FIG. 4D, FIG. 4E, and FIG. 4F depict a comparison of NP properties of siRNA-liposomes and siRNA-leukosomes. Size (FIG. 4A) and polydispersity index (PDI) (FIG. 4B) measurements revealed significant differences between siRNA-liposomes and siRNA-leukosomes, especially after dialysis. Both NPs maintained an overall negative charge in their zeta potential (FIG. 4C). FIG. 4D shows visual inspection of the collected samples further corroborated the differences between liposomes and leukosome prior to filtration. FIG. 4E shows quantification of siRNA loss demonstrated the significant reduction of siRNA in the leukosomes during the filtration step. n=3. Results are shown as means±SEM. 2-way ANOVA followed by Sidak's multiple comparison test was used to determine statistical significance in A-B. Welch's t-test was used to determine statistical significance in F. P value≤0.05 among means was considered as statistically significant.

FIG. 5A, FIG. 5B, and FIG. 5C depict the effect of removing PEG on NP physicochemical properties. Evaluation of size (FIG. 5A), polydispersity index (PDI) (FIG. 5B), and zeta potential (FIG. 5C) of siRNA-liposomes and siRNA-leukosomes with and without PEG demonstrated the significant changes in NP properties resulting from the PEG removal. n=3. Results are shown as means±SEM. 2-way ANOVA followed by Sidak's multiple comparison test was used to determine statistical significance. P value≤0.05 among means was considered as statistically significant.

FIG. 6A, FIG. 6B, FIG. 6C, and FIG. 6D depict the effect of increasing flow rate on NP physicochemical properties. Size (FIGS. 6A-6B) and polydispersity (PDI) (FIGS. 6C-6D) measurements of NPs synthesized with increasing flow rate (FR) indicated minimal changes in these NP characteristics both liposomes and leukosomes. n=3-12. Results are shown as means±SEM. 2-way ANOVA followed by Tukey's multiple comparison test was used to determine statistical significance. P value≤0.05 among means was considered as statistically significant.

FIG. 7A and FIG. 7B depict a cytotoxicity profile of siRNA-leukosomes with different PEG ratios on 4T1 cells. Cell viability analysis following treatment of 4T1 cells with leukosomes of mid PEG percentage (FIG. 7A) and high PEG percentage (FIG. 7B) for up to 72 h indicated reduction of viability in concentrations above 500 μM and 250 μM, respectively. n=4. Results are shown as means±SEM.

FIG. 8A and FIG. 8B depict a knockdown efficiency of siRNA-NPs with different PEG ratios on 4T1 cells. Change in STAT3 mRNA expression following treatment with NPs of mid (FIG. 8A) and high (FIG. 8B) PEG ratios corroborated the ability of the synthesized NPs to achieve knockdown efficiencies comparable to the Lipofectamine control, with only STAT3 siRNA-liposomes of high PEG content inducing minimal RNA knockdown. n=4. Results are shown as means±SEM. 2-way ANOVA followed by Dunnett's multiple comparison test was used to determine statistical significance. P value≤0.05 among means was considered as statistically significant.

FIG. 9A, FIG. 9B, FIG. 9C, and FIG. 9D depict a biodistribution profile of siRNA-NPs with varying PEG ratios. Ex vivo organ analysis for NP accumulation at 1 h (FIGS. 9A-9B) and 6 h (FIGS. 9C-9D) indicated differences in the overall liver and tumor accumulation profiles between the siRNA-NPs of mid (FIG. 9A, FIG. 9C) and high (FIG. 9B, FIG. 9D) PEG concentration. n=3-5. Results are shown as means±SEM.

FIG. 10A, FIG. 10B, FIG. 10C, and FIG. 10D show tumor and liver accumulation of siRNA-NPs with varying PEG ratios. Ex vivo organ analysis for siRNA-NPs of (FIG. 10A, FIG. 10C) mid and (FIG. 10B, FIG. 10D) high PEG concentration NP accumulation in the (FIG. 10A-FIG. 10B) tumor and (FIG. 10C-FIG. 10D) liver. n=3-5. Results are shown as means±SEM.

FIG. 11 depicts a schematic of the leukosome-LNPs.

FIG. 12A, FIG. 12B, FIG. 12C, and FIG. 12D depict testing results of iteration 1 for siRNA encapsulation.

FIG. 13A, FIG. 13B, FIG. 13C, and FIG. 13D depict testing results of iteration 2 for siRNA encapsulation.

FIG. 14 depicts day 0 post synthesis of iteration 3 for siRNA encapsulation.

FIG. 15A, FIG. 15B, FIG. 15C, and FIG. 15D depict stability results of iteration 3 for siRNA encapsulation.

FIG. 16A and FIG. 16B depicts in vitro tests for siRPL39 Leukosomes-LNPs.

FIG. 17A, FIG. 17B, and FIG. 17C depict in vivo downregulation tests for siRPL39 Leukosomes-LNPs.

FIG. 18A and FIG. 18B depict in vivo targeting tests for siRPL39 Leukosomes-LNPs.

FIG. 19A, FIG. 19B, FIG. 19C, and FIG. 19D depict testing results of iteration 1 for mRNA encapsulation.

FIG. 20A, FIG. 20B, FIG. 20C, and FIG. 20D depict in vivo biodistribution of Leukosome-LNPs.

FIG. 21A and FIG. 21B depict in vivo biodistribution and targeting of iteration 2 for mRNA encapsulation.

FIG. 22A and FIG. 22B depict in vivo biodistribution and targeting of iteration 2 for mRNA encapsulation.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

In this specification and in the claims that follow, reference will be made to a number of terms, which shall be defined to have the following meanings:

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “a composition”, or “a cancer”, includes, but is not limited to, two or more such compounds, compositions, or cancers, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biocompatible” material is a synthetic or natural material used to replace part of a living system or to function in intimate contact with living tissue. Biocompatible materials are intended to interface with biological systems to evaluate, treat, augment, or replace any tissue, organ, or function of the body. The biocompatible material has the ability to perform with an appropriate host response in a specific application and does not have toxic or injurious effects on biological systems. One example of a biocompatible material can be a biocompatible ceramic.

As used herein, “biomimetic” shall mean a resemblance of a synthesized material to a substance that occurs naturally in a human body and which is not rejected by (e.g., does not cause an adverse reaction in) the human body.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example from a pH of about 5 to 7.

As used herein, the term “carrier” is intended to include any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

As used herein, the term “effective amount” refers to an amount that is sufficient to achieve the desired modification of a physical property of the composition or material. For example, an “effective amount” of a monomer refers to an amount that is sufficient to achieve the desired improvement in the property modulated by the formulation component, e.g. desired antioxidant release rate or viscoelasticity. The specific level in terms of wt % in a composition required as an effective amount will depend upon a variety of factors including the amount and type of monomer, amount and type of polymer, e.g., acrylamide, amount of antioxidant, and desired release kinetics.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed drug delivery composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an ophthalmological disorder. The effect can be therapeutic in terms of a partial or complete cure of a disease, condition, symptom or adverse effect attributed to the disease, disorder, or condition. The term “treatment” as used herein can include any treatment of ophthalmological disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion polynucleotides also are contemplated herein. A “fusion polynucleotide” refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

The term “mismatched” or “mismatched target sequence” refers to an off-target sequence that is not perfectly complementary to the first DNA sequence or the second DNA sequence of the chimeric deoxyribonucleic acid described herein. The dual retargeted DNA may have at least one mismatch, but can also have 2, 3, 4, 5, 6 or 7 or more mismatched nucleotides to the off-target sequence.

siRNA-Containing Biomimetic Proteolipid Nanovesicles

In an aspect, provided is a biomimetic proteolipid nanovesicle, including: an ionizable or cationic lipid; a phosphocholine-based phospholipid; a cholesterol; a leukocyte membrane protein; and an siRNA encapsulated by the biomimetic proteolipid nanovesicle; wherein the biomimetic proteolipid nanovesicle can have a lipid-to-protein ratio of from about 1:65 to about 1:85 by weight.

In some aspects, the biomimetic proteolipid nanovesicle can have a lipid-to-protein ratio of at least about 1:50 (e.g., at least about 1:55, at least about 1:60, at least about 1:65, at least about 1:66, at least about 1:67, at least about 1:68, at least about 1:69, at least about 1:70, at least about 1:71, at least about 1:72, at least about 1:73, at least about 1:74, at least about 1:75, at least about 1:76, at least about 1:77, at least about 1:78, at least about 1:79, at least about 1:80, at least about 1:81, at least about 1:82, at least about 1:83, at least about 1:84, at least about 1:85, at least about 1:90, at least about 1:95, at least about 1:100) by weight. In some aspects, the biomimetic proteolipid nanovesicle can have a lipid-to-protein ratio of up to about 1:100 (e.g., up to about 1:95, up to about 1:90, up to about 1:85, up to about 1:84, up to about 1:83, up to about 1:82, up to about 1:81, up to about 1:80, up to about 1:79, up to about 1:78, up to about 1:77, up to about 1:76, up to about 1:75, up to about 1:74, up to about 1:73, up to about 1:72, up to about 1:71, up to about 1:70, up to about 1:69, up to about 1:68, up to about 1:67, up to about 1:66, up to about 1:65, up to about 1:60, up to about 1:55, up to about 1:50) by weight. In some aspects, the biomimetic proteolipid nanovesicle can have a lipid-to-protein ratio of about 1:50, about 1:55, about 1:60, about 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:90, about 1:95, or about 1:100 by weight.

It is considered that the biomimetic proteolipid nanovesicle can have a lipid-to-protein ratio ranging from any of the minimum values described above to any of the maximum values described above. For example, in some aspects, the biomimetic proteolipid nanovesicle can have a lipid-to-protein ratio of from about 1:65 to about 1:85 (e.g., from about 1:66 to about 1:84, from about 1:67 to about 1:83, from about 1:68 to about 1:82, from about 1:69 to about 1:81, from about 1:70 to about 1:80, from about 1:71 to about 1:79, from about 1:72 to about 1:78, from about 1:73 to about 1:77, from about 1:74 to about 1:76, from about 1:65 to about 1:75, from about 1:66 to about 1:74, from about 1:67 to about 1:73, from about 1:68 to about 1:72, from about 1:69 to about 1:71, from about 1:75 to about 1:85, from about 1:76 to about 1:84, from about 1:77 to about 1:83, from about 1:78 to about 1:82, from about 1:79 to about 1:81) by weight. In some aspects, the biomimetic proteolipid nanovesicle can have a lipid-to-protein ratio of from about 1:50 to about 1:100 by weight.

In some aspects, the ionizable or cationic lipid can be selected from the group consisting of DLin-MC3-DMA, SM-102, ALC-0315, 1,2-dimyristoyl-3-dimethylammonium-propane (DAP), 1,2-dioleoyl-3-trimethylammonium propane (DOTAP), C12-200, 5A2-SC8, and any combination thereof.

In some aspects, the biomimetic proteolipid nanovesicle can further include at least about 40 molar % (e.g., at least about 42 molar %, at least about 44 molar %, at least about 46 molar %, at least about 48 molar %, at least about 50 molar %, at least about 52 molar %, at least about 54 molar %, at least about 56 molar %, at least about 58 molar %, at least about 60 molar %) of the ionizable or cationic lipid. In some aspects, the biomimetic proteolipid nanovesicle can further include up to about 60 molar % (e.g., up to about 58 molar %, up to about 56 molar %, up to about 54 molar %, up to about 52 molar %, up to about 50 molar %, up to about 48 molar %, up to about 46 molar %, up to about 44 molar %, up to about 42 molar %, up to about 40 molar %) of the ionizable or cationic lipid. In some aspects, the biomimetic proteolipid nanovesicle can further include about 40 molar %, about 42 molar %, about 44 molar %, about 46 molar %, about 48 molar %, about 50 molar %, about 52 molar %, about 54 molar %, about 56 molar %, about 58 molar %, or about 60 molar % of the ionizable or cationic lipid.

It is considered that the biomimetic proteolipid nanovesicle can further include an amount of the ionizable or cationic lipid ranging from any of the minimum values described above to any of the maximum values described above. For example, in some aspects, the biomimetic proteolipid nanovesicle can include from about 40 molar % to about 60 molar % (e.g., from about 42 molar % to about 58 molar %, from about 44 molar % to about 56 molar %, from about 46 molar % to about 54 molar %, from about 48 molar % to about 52 molar %, from about 40 molar % to about 50 molar %, from about 42 molar % to about 48 molar %, from about 44 molar % to about 46 molar %, from about 50 molar % to about 60 molar %, from about 52 molar % to about 58 molar %, from about 54 molar % to about 56 molar %) of the ionizable or cationic lipid.

In some aspects, the phosphocholine-based phospholipid can be selected from the group consisting of phosphatidylcholine, egg phosphatidic acid, 1,2-dioleoyl-sn-glycerophosphocholine (DOPC), 1,2-diolyl-sn-lycerophosphoethanolamine (DOPE), 1,2-dipalmitoyl-sn-glycerophosphocholine (DPPC), 1,2-distearoyl-sn-glycerophosphocholine (DSPC), L-ct-phosphatidylserine (PS), 1,2-dioleoyl-sn-glycero-3-phospho-(1′-rac-glycerol) (DOPG), and any combination thereof.

In some aspects, the biomimetic proteolipid nanovesicle can include at least about 7 molar % (e.g., at least about 7.5 molar %, at least about 8 molar %, at least about 8.5 molar %, at least about 9 molar %, at least about 9.5 molar %, at least about 10 molar %, at least about 10.5 molar %, at least about 11 molar %, at least about 11.5 molar %, at least about 12 molar %, at least about 12.5 molar %, at least about 13 molar %, at least about 13.5 molar %, at least about 14 molar %, at least about 14.5 molar %, at least about 15 molar %) of the phosphocholine-based phospholipid. In some aspects, the biomimetic proteolipid nanovesicle can include up to about 15 molar % (e.g., up to about 14.5 molar %, up to about 14 molar %, up to about 13.5 molar %, up to about 13 molar %, up to about 12.5 molar %, up to about 12 molar %, up to about 11.5 molar %, up to about 11 molar %, up to about 10.5 molar %, up to about 10 molar %, up to about 9.5 molar %, up to about 9 molar %, up to about 8.5 molar %, up to about 8 molar %, up to about 7.5 molar %, up to about 7 molar %) of the phosphocholine-based phospholipid. In some aspects, the biomimetic proteolipid nanovesicle can include about 7 molar %, about 7.5 molar %, about 8 molar %, about 8.5 molar %, about 9 molar %, about 9.5 molar %, about 10 molar %, about 10.5 molar %, about 11 molar %, about 11.5 molar %, about 12 molar %, about 12.5 molar %, about 13 molar %, about 13.5 molar %, about 14 molar %, about 14.5 molar %, or about 15 molar % of the phosphocholine-based phospholipid.

It is considered that the biomimetic proteolipid nanovesicle can further include an amount of the phosphocholine-based phospholipid ranging from any of the minimum values described above to any of the maximum values described above. For example, in some aspects, the biomimetic proteolipid nanovesicle can include from about 7 molar % to about 15 molar % (e.g., from about 7.5 molar % to about 14.5 molar %, from about 8 molar % to about 14 molar %, from about 8.5 molar % to about 13.5 molar %, from about 9 molar % to about 13 molar %, from about 9.5 molar % to about 12.5 molar %, from about 10 molar % to about 12 molar %, from about 10.5 molar % to about 11.5 molar %, from about 7 molar % to about 11 molar %, from about 7.5 molar % to about 10.5 molar %, from about 8 molar % to about 10 molar %, from about 8.5 molar % to about 9.5 molar %, from about 11 molar % to about 15 molar %, from about 11.5 molar % to about 14.5 molar %, from about 12 molar % to about 14 molar %, from about 12.5 molar % to about 13.5 molar %) of the phosphocholine-based phospholipid.

In some aspects, the biomimetic proteolipid nanovesicle can include at least about 30 molar % (e.g., at least about 30.5 molar %, at least about 31 molar %, at least about 31.5 molar %, at least about 32 molar %, at least about 32.5 molar %, at least about 33 molar %, at least about 33.5 molar %, at least about 34 molar %, at least about 34.5 molar %, at least about 35 molar %, at least about 35.5 molar %, at least about 36 molar %, at least about 36.5 molar %, at least about 37 molar %, at least about 37.5 molar %, at least about 38 molar %, at least about 38.5 molar %, at least about 39 molar %, at least about 39.5 molar %, at least about 40 molar %) of the cholesterol. In some aspects, the biomimetic proteolipid nanovesicle can include up to about 40 molar % (e.g., up to about 39.5 molar %, up to about 39 molar %, up to about 38.5 molar %, up to about 38 molar %, up to about 37.5 molar %, up to about 37 molar %, up to about 36.5 molar %, up to about 36 molar %, up to about 35.5 molar %, up to about 35 molar %, up to about 34.5 molar %, up to about 34 molar %, up to about 33.5 molar %, up to about 33 molar %, up to about 32.5 molar %, up to about 32 molar %, up to about 31.5 molar %, up to about 31 molar %, up to about 30.5 molar %, up to about 30 molar %) of the cholesterol. In some aspects, the biomimetic proteolipid nanovesicle can include about 30 molar %, about 30.5 molar %, about 31 molar %, about 31.5 molar %, about 32 molar %, about 32.5 molar %, about 33 molar %, about 33.5 molar %, about 34 molar %, about 34.5 molar %, about 35 molar %, about 35.5 molar %, about 36 molar %, about 36.5 molar %, about 37 molar %, about 37.5 molar %, about 38 molar %, about 38.5 molar %, about 39 molar %, about 39.5 molar %, or about 40 molar % of the cholesterol.

It is considered that the biomimetic proteolipid nanovesicle can further include an amount of the cholesterol ranging from any of the minimum values described above to any of the maximum values described above. For example, in some aspects, the biomimetic proteolipid nanovesicle can include from about 30 molar % to about 40 molar % (e.g., from about 30.5 molar % to about 39.5 molar %, from about 31 molar % to about 39 molar %, from about 31.5 molar % to about 38.5 molar %, from about 32 molar % to about 38 molar %, from about 32.5 molar % to about 37.5 molar %, from about 33 molar % to about 37 molar %, from about 33.5 molar % to about 36.5 molar %, from about 34 molar % to about 36 molar %, from about 34.5 molar % to about 35.5 molar %, from about 30 molar % to about 35 molar %, from about 30.5 molar % to about 34.5 molar %, from about 31 molar % to about 34 molar %, from about 31.5 molar % to about 33.5 molar %, from about 32 molar % to about 33 molar %, from about 35 molar % to about 40 molar %, from about 35.5 molar % to about 39.5 molar %, from about 36 molar % to about 39 molar %, from about 36.5 molar % to about 38.5 molar %, from about 37 molar % to about 38 molar %) of the cholesterol.

In some aspects, the biomimetic proteolipid nanovesicle can further include a PEGylated lipid. In some aspects, the PEGylated lipid can be selected from the group consisting of DMG-PEG2000, ALC-0159, DSPE-PEG2000, DOPE-PEG2000, 18:1 PEG1000-PE, and any combination thereof.

In some aspects, the biomimetic proteolipid nanovesicle can further include at least about 1 molar % (e.g., at least about 1.5 molar %, at least about 2 molar %, at least about 2.5 molar %, at least about 3 molar %, at least about 3.5 molar %, at least about 4 molar %, at least about 4.5 molar %, at least about 5 molar %, at least about 5.5 molar %, at least about 6 molar %, at least about 6.5 molar %, at least about 7 molar %, at least about 7.5 molar %, at least about 8 molar %, at least about 8.5 molar %, at least about 9 molar %, at least about 9.5 molar %, at least about 10 molar %) of the PEGylated lipid. In some aspects, the biomimetic proteolipid nanovesicle can further include up to about 10 molar % (e.g., up to about 9.5 molar %, up to about 9 molar %, up to about 8.5 molar %, up to about 8 molar %, up to about 7.5 molar %, up to about 7 molar %, up to about 6.5 molar %, up to about 6 molar %, up to about 5.5 molar %, up to about 5 molar %, up to about 4.5 molar %, up to about 4 molar %, up to about 3.5 molar %, up to about 3 molar %, up to about 2.5 molar %, up to about 2 molar %, up to about 1.5 molar %, up to about 1 molar %) of the PEGylated lipid. In some aspects, the biomimetic proteolipid nanovesicle can include about 1 molar %, about 1.5 molar %, about 2 molar %, about 2.5 molar %, about 3 molar %, about 3.5 molar %, about 4 molar %, about 4.5 molar %, about 5 molar %, about 5.5 molar %, about 6 molar %, about 6.5 molar %, about 7 molar %, about 7.5 molar %, about 8 molar %, about 8.5 molar %, about 9 molar %, about 9.5 molar %, or about 10 molar % of the PEGylated lipid.

It is considered that the biomimetic proteolipid nanovesicle can further include an amount of the PEGylated lipid ranging from any of the minimum values described above to any of the maximum values described above. For example, in some aspects, the biomimetic proteolipid nanovesicle can include from about 1 molar % to about 10 molar % (e.g., from about 1.5 molar % to about 9.5 molar %, from about 2 molar % to about 9 molar %, from about 2.5 molar % to about 8.5 molar %, from about 3 molar % to about 8 molar %, from about 3.5 molar % to about 7.5 molar %, from about 4 molar % to about 7 molar %, from about 4.5 molar % to about 6.5 molar %, from about 5 molar % to about 6 molar %, from about 1 molar % to about 5.5. molar %, from about 1.5 molar % to about 5 molar %, from about 2 molar % to about 4.5 molar %, from about 2.5 molar % to about 4 molar %, from about 3 molar % to about 3.5 molar %, from about 5.5. molar % to about 10 molar %, from about 6 molar % to about 9.5 molar %, from about 6.5 molar % to about 9 molar %, from about 7 molar % to about 8.5 molar %, from about 7.5 molar % to about 8 molar %) of the PEGylated lipid.

In some aspects, the leukocyte membrane protein can be derived from a leukocyte plasma membrane. In some such aspects, the leukocyte plasma membrane can be a human leukocyte plasma membrane. In other such aspects, the leukocyte plasma membrane can be derived from a murine leukocyte plasma membrane. In other aspects, the leukocyte membrane protein can be a synthetic recombinant protein.

In some aspects, the leukocyte membrane protein can be lymphocyte function-associated antigen 1 (LFA-1), CD11, CD45, CD47, or any combination thereof. In some aspects, the leukocyte membrane protein can include some or all of the peptides present in a leukocyte plasma membrane (e.g., a human leukocyte plasma membrane or a murine leukocyte plasma membrane).

The biomimetic proteolipid nanovesicles can, in some aspects, be further defined by an N/P ratio. As used herein, “N/P ratio” refers to the nitrogen-to-phosphate ratio, where “N” represents the nitrogen atoms from the ionizable or cationic lipid, which typically contains amine groups, and “P” refers to the phosphate groups in the nucleic acid molecules, such as the phosphate backbone of siRNA or mRNA. This ratio can ensure the stability, charge balance, and transfection efficiency of the lipid nanoparticles. Its optimization can help achieve optimal encapsulation efficiency in the context of different nucleic acid payloads.

In some aspects, the biomimetic proteolipid nanovesicle can further include an N/P ratio of at least about 1 (e.g., at least about 1.25, at least about 1.5, at least about 1.75, at least about 2, at least about 2.25, at least about 2.5, at least about 2.75, at least about 3, at least about 3.25, at least about 3.5, at least about 3.75, at least about 4, at least about 4.25, at least about 4.5, at least about 4.75, at least about 5, at least about 5.25, at least about 5.5, at least about 5.75, at least about 6). In some aspects, the biomimetic proteolipid nanovesicle can further include an N/P ratio of up to about 6 (e.g., up to about 5.75, up to about 5.5, up to about 5.25, up to about 5, up to about 4.75, up to about 4.5, up to about 4.25, up to about 4, up to about 3.75, up to about 3.5, up to about 3.25, up to about 3, up to about 2.75, up to about 2.5, up to about 2.25, up to about 2, up to about 1.75, up to about 1.5, up to about 1.25, up to about 1). In some aspects, the biomimetic proteolipid nanovesicle can further include an N/P ratio of about 1, about 1.25, about 1.5, about 1.75, about 2, about 2.25, about 2.5, about 2.75, about 3, about 3.25, about 3.5, about 3.75, about 4, about 4.25, about 4.5, about 4.75, about 5, about 5.25, about 5.5, about 5.75, or about 6.

It is considered that the biomimetic proteolipid nanovesicle can further include an N/P ratio ranging from any of the minimum values described above to any of the maximum values described above. For example, in some aspects, the biomimetic proteolipid nanovesicle can further include an N/P ratio of from about 1 to about 6 (e.g., from about 1.25 to about 5.75, from about 1.5 to about 5.5, from about 1.75 to about 5.25, from about 2 to about 5, from about 2.25 to about 4.75, from about 2.5 to about 4.5, from about 2.75 to about 4.25, from about 3 to about 4, from about 3.25 to about 3.75, from about 1 to about 3.5, from about 1.25 to about 3.25, from about 1.5 to about 3, from about 1.75 to about 2.75, from about 2 to about 2.5, from about 3.5 to about 6, from about 3.75 to about 5.75, from about 4 to about 5.5, from about 4.25 to about 5.25, from about 4.5 to about 5).

In some aspects, the biomimetic proteolipid nanovesicle can have a diameter of at least about 50 nm (e.g., at least about 60 nm, at least about 70 nm, at least about 80 nm, at least about 90 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm). In some aspects, the biomimetic proteolipid nanovesicle can have a diameter of up to about 200 nm (e.g., up to about 190 nm, up to about 180 nm, up to about 170 nm, up to about 160 nm, up to about 150 nm, up to about 140 nm, up to about 130 nm, up to about 120 nm, up to about 110 nm, up to about 100 nm, up to about 90 nm, up to about 80 nm, up to about 70 nm, up to about 60 nm, up to about 50 nm). In some aspects, the biomimetic proteolipid nanovesicle can have a diameter of about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm.

It is considered that the biomimetic proteolipid nanovesicle can have a diameter ranging from any of the minimum values described above to any of the maximum values described above. For example, in some aspects, the biomimetic proteolipid nanovesicle can have a diameter of from about 50 nm to about 200 nm (e.g., from about 60 nm to about 190 nm, from about 70 nm to about 180 nm, from about 80 nm to about 170 nm, from about 90 nm to about 160 nm, from about 100 nm to about 150 nm, from about 110 nm to about 140 nm, from about 120 nm to about 130 nm, from about 50 nm to about 130 nm, from about 60 nm to about 120 nm, from about 70 nm to about 110 nm, from about 80 nm to about 100 nm, from about 120 nm to about 200 nm, from about 130 nm to about 190 nm, from about 140 nm to about 180 nm, from about 150 nm to about 170 nm).

In some aspects, the siRNA can be targeted to treat a cancer, inflammation, an infectious disease, or a genetic disease or disorder.

mRNA-Containing Biomimetic Proteolipid Nanovesicles

In an aspect, provided is a biomimetic proteolipid nanovesicle, including: an ionizable or cationic lipid; a phosphocholine-based phospholipid; a cholesterol; a leukocyte membrane protein; and an mRNA encapsulated by the biomimetic proteolipid nanovesicle; wherein the biomimetic proteolipid nanovesicle can have a lipid-to-protein ratio of from about 1:80 to about 1:100 by weight.

In some aspects, the biomimetic proteolipid nanovesicle can have a lipid-to-protein ratio of at least about 1:80 (e.g., at least about at least about 1:81, at least about 1:82, at least about 1:83, at least about 1:84, at least about 1:85, at least about 1:86, at least about 1:87, at least about 1:88, at least about 1:89, at least about 1:90, at least about 1:91, at least about 1:92, at least about 1:93, at least about 1:94, at least about 1:95, at least about 1:96, at least about 1:97, at least about 1:98, at least about 1:99, at least about 1:100) by weight. In some aspects, the biomimetic proteolipid nanovesicle can have a lipid-to-protein ratio of up to about 1:100 (e.g., up to about 1:99, up to about 1:98, up to about 1:97, up to about 1:96, up to about 1:95, up to about 1:94, up to about 1:93, up to about 1:92, up to about 1:91, up to about 1:90, up to about 1:89, up to about 1:88, up to about 1:87, up to about 1:86, up to about 1:85, up to about 1:84, up to about 1:83, up to about 1:82, up to about 1:81, up to about 1:80) by weight. In some aspects, the biomimetic proteolipid nanovesicle can have a lipid-to-protein ratio of about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, or about 1:100 by weight.

It is considered that the biomimetic proteolipid nanovesicle can have a lipid-to-protein ratio ranging from any of the minimum values described above to any of the maximum values described above. For example, in some aspects, the biomimetic proteolipid nanovesicle can have a lipid-to-protein ratio of from about 1:80 to about 1:100 (e.g., from about 1:81 to about 1:99, from about 1:82 to about 1:98, from about 1:83 to about 1:97, from about 1:84 to about 1:96, from about 1:85 to about 1:95, from about 1:86 to about 1:94, from about 1:87 to about 1:93, from about 1:88 to about 1:92, from about 1:89 to about 1:91, from about 1:80 to about 1:90, from about 1:81 to about 1:89, from about 1:82 to about 1:88, from about 1:83 to about 1:87, from about 1:84 to about 1:86, from about 1:90 to about 1:100, from about 1:91 to about 1:99, from about 1:92 to about 1:98, from about 1:93 to about 1:97, from about 1:94 to about 1:96) by weight.

In some aspects, at least one ionizable or cationic lipid can be selected from the group consisting of DLin-MC3-DMA, SM-102, ALC-0315, and any combination thereof.

In some aspects, the biomimetic proteolipid nanovesicle can further include at least about 40 molar % (e.g., at least about 42 molar %, at least about 44 molar %, at least about 46 molar %, at least about 48 molar %, at least about 50 molar %, at least about 52 molar %, at least about 54 molar %, at least about 56 molar %, at least about 58 molar %, at least about 60 molar %) of the ionizable or cationic lipid. In some aspects, the biomimetic proteolipid nanovesicle can further include up to about 60 molar % (e.g., up to about 58 molar %, up to about 56 molar %, up to about 54 molar %, up to about 52 molar %, up to about 50 molar %, up to about 48 molar %, up to about 46 molar %, up to about 44 molar %, up to about 42 molar %, up to about 40 molar %) of the ionizable or cationic lipid. In some aspects, the biomimetic proteolipid nanovesicle can further include about 40 molar %, about 42 molar %, about 44 molar %, about 46 molar %, about 48 molar %, about 50 molar %, about 52 molar %, about 54 molar %, about 56 molar %, about 58 molar %, or about 60 molar % of the ionizable or cationic lipid.

It is considered that the biomimetic proteolipid nanovesicle can further include an amount of the ionizable or cationic lipid ranging from any of the minimum values described above to any of the maximum values described above. For example, in some aspects, the biomimetic proteolipid nanovesicle can include from about 40 molar % to about 60 molar % (e.g., from about 42 molar % to about 58 molar %, from about 44 molar % to about 56 molar %, from about 46 molar % to about 54 molar %, from about 48 molar % to about 52 molar %, from about 40 molar % to about 50 molar %, from about 42 molar % to about 48 molar %, from about 44 molar % to about 46 molar %, from about 50 molar % to about 60 molar %, from about 52 molar % to about 58 molar %, from about 54 molar % to about 56 molar %) of the ionizable or cationic lipid.

In some aspects, the phosphocholine-based phospholipid can be selected from the group consisting of phosphatidylcholine, egg phosphatidic acid, 1,2-dioleoyl-sn-glycerophosphocholine (DOPC), 1,2-diolyl-sn-lycerophosphoethanolamine (DOPE), 1,2-dipalmitoyl-sn-glycerophosphocholine (DPPC), 1,2-distearoyl-sn-glycerophosphocholine (DSPC), L-α-phosphatidylserine (PS), 1,2-dioleoyl-sn-glycero-3-phospho-(1′-rac-glycerol) (DOPG), and any combination thereof.

In some aspects, the biomimetic proteolipid nanovesicle can further include at least about 7 molar % (e.g., at least about 7.5 molar %, at least about 8 molar %, at least about 8.5 molar %, at least about 9 molar %, at least about 9.5 molar %, at least about 10 molar %, at least about 10.5 molar %, at least about 11 molar %, at least about 11.5 molar %, at least about 12 molar %, at least about 12.5 molar %, at least about 13 molar %, at least about 13.5 molar %, at least about 14 molar %, at least about 14.5 molar %, at least about 15 molar %) of the phosphocholine-based phospholipid. In some aspects, the biomimetic proteolipid nanovesicle can further include up to about 15 molar % (e.g., up to about 14.5 molar %, up to about 14 molar %, up to about 13.5 molar %, up to about 13 molar %, up to about 12.5 molar %, up to about 12 molar %, up to about 11.5 molar %, up to about 11 molar %, up to about 10.5 molar %, up to about 10 molar %, up to about 9.5 molar %, up to about 9 molar %, up to about 8.5 molar %, up to about 8 molar %, up to about 7.5 molar %, up to about 7 molar %) of the phosphocholine-based phospholipid. In some aspects, the biomimetic proteolipid nanovesicle can further include about 7 molar %, about 7.5 molar %, about 8 molar %, about 8.5 molar %, about 9 molar %, about 9.5 molar %, about 10 molar %, about 10.5 molar %, about 11 molar %, about 11.5 molar %, about 12 molar %, about 12.5 molar %, about 13 molar %, about 13.5 molar %, about 14 molar %, about 14.5 molar %, about 15 molar % of the phosphocholine-based phospholipid.

It is considered that the biomimetic proteolipid nanovesicle can further include an amount of the phosphocholine-based phospholipid ranging from any of the minimum values described above to any of the maximum values described above. For example, in some aspects, the biomimetic proteolipid nanovesicle can further include from about 7 molar % to about 15 molar % (e.g., from about 7.5 molar % to about 14.5 molar %, from about 8 molar % to about 14 molar %, from about 8.5 molar % to about 13.5 molar %, from about 9 molar % to about 13 molar %, from about 9.5 molar % to about 12.5 molar %, from about 10 molar % to about 11 molar %, from about 7 molar % to about 10.5 molar %, from about 7.5 molar % to about 10 molar %, from about 8 molar % to about 9.5 molar %, from about 8.5 molar % to about 9 molar %, from about 10.5 molar % to about 15 molar %, from about 11 molar % to about 14.5 molar %, from about 11.5 molar % to about 14 molar %, from about 12 molar % to about 13.5 molar %, from about 12.5 molar % to about 13 molar %) of the phosphocholine-based phospholipid.

In some aspects, the biomimetic proteolipid nanovesicle can further include at least about 35 molar % (e.g., at least about 35.5 molar %, at least about 36 molar %, at least about 36.5 molar %, at least about 37 molar %, at least about 37.5 molar %, at least about 38 molar %, at least about 38.5 molar %, at least about 39 molar %, at least about 39.5 molar %, at least about 40 molar %, at least about 40.5 molar %, at least about 41 molar %, at least about 41.5 molar %, at least about 42 molar %, at least about 42.5 molar %, at least about 43 molar %, at least about 43.5 molar %, at least about 44 molar %, at least about 44.5 molar %, at least about 45 molar %) of the cholesterol. In some aspects, the biomimetic proteolipid nanovesicle can further include up to about 45 molar % (e.g., up to about 44.5 molar %, up to about 44 molar %, up to about 43.5 molar %, up to about 43 molar %, up to about 42.5 molar %, up to about 42 molar %, up to about 41.5 molar %, up to about 41 molar %, up to about 40.5 molar %, up to about 40 molar %, up to about 39.5 molar %, up to about 39 molar %, up to about 38.5 molar %, up to about 38 molar %, up to about 37.5 molar %, up to about 37 molar %, up to about 36.5 molar %, up to about 36 molar %, up to about 35.5 molar %, up to about 35 molar %) of the cholesterol. In some aspects, the biomimetic proteolipid nanovesicle can further include about 35 molar %, about 35.5 molar %, about 36 molar %, about 36.5 molar %, about 37 molar %, about 37.5 molar %, about 38 molar %, about 38.5 molar %, about 39 molar %, about 39.5 molar %, about 40 molar %, about 40.5 molar %, about 41 molar %, about 41.5 molar %, about 42 molar %, about 42.5 molar %, about 43 molar %, about 43.5 molar %, about 44 molar %, about 44.5 molar %, or about 45 molar % of the cholesterol.

It is considered that the biomimetic proteolipid nanovesicle can further include an amount of the cholesterol ranging from any of the minimum values described above to any of the maximum values described above. For example, in some aspects, the biomimetic proteolipid nanovesicle can further include from about 35 molar % to about 45 molar % (e.g., from about 35.5 molar % to about 44.5 molar %, from about 36 molar % to about 44 molar %, from about 36.5 molar % to about 43.5 molar %, from about 37 molar % to about 43 molar %, from about 37.5 molar % to about 42.5 molar %, from about 38 molar % to about 42 molar %, from about 38.5 molar % to about 41.5 molar %, from about 39 molar % to about 41 molar %, from about 39.5 molar % to about 40.5 molar %, from about 35 molar % to about 40 molar %, from about 35.5 molar % to about 39.5 molar %, from about 36 molar % to about 39 molar %, from about 36.5 molar %, from about 38.5 molar %, from about 37 molar % to about 38 molar %, from about 40 molar % to about 45 molar %, from about 40.5 molar % to about 44.5 molar %, from about 41 molar % to about 44 molar %, from about 41.5 molar % to about 43.5 molar %, from about 42 molar % to about 43 molar %) of the cholesterol.

In some aspects, the biomimetic proteolipid nanovesicle can further include a PEGylated lipid. In some aspects, the PEGylated lipid can be selected from the group consisting of DMG-PEG2000, ALC-0159, and any combination thereof.

In some aspects, the biomimetic proteolipid nanovesicle can further include at least about 1 molar % (e.g., at least about 1.5 molar %, at least about 2 molar %, at least about 2.5 molar %, at least about 3 molar %, at least about 3.5 molar %, at least about 4 molar %, at least about 4.5 molar %, at least about 5 molar %, at least about 5.5 molar %, at least about 6 molar %, at least about 6.5 molar %, at least about 7 molar %, at least about 7.5 molar %, at least about 8 molar %, at least about 8.5 molar %, at least about 9 molar %, at least about 9.5 molar %, at least about 10 molar %) of the PEGylated lipid. In some aspects, the biomimetic proteolipid nanovesicle can further include up to about 10 molar % (e.g., up to about 9.5 molar %, up to about 9 molar %, up to about 8.5 molar %, up to about 8 molar %, up to about 7.5 molar %, up to about 7 molar %, up to about 6.5 molar %, up to about 6 molar %, up to about 5.5 molar %, up to about 5 molar %, up to about 4.5 molar %, up to about 4 molar %, up to about 3.5 molar %, up to about 3 molar %, up to about 2.5 molar %, up to about 2 molar %, up to about 1.5 molar %, up to about 1 molar %) of the PEGylated lipid. In some aspects, the biomimetic proteolipid nanovesicle can include about 1 molar %, about 1.5 molar %, about 2 molar %, about 2.5 molar %, about 3 molar %, about 3.5 molar %, about 4 molar %, about 4.5 molar %, about 5 molar %, about 5.5 molar %, about 6 molar %, about 6.5 molar %, about 7 molar %, about 7.5 molar %, about 8 molar %, about 8.5 molar %, about 9 molar %, about 9.5 molar %, or about 10 molar % of the PEGylated lipid.

It is considered that the biomimetic proteolipid nanovesicle can further include an amount of the PEGylated lipid ranging from any of the minimum values described above to any of the maximum values described above. For example, in some aspects, the biomimetic proteolipid nanovesicle can include from about 1 molar % to about 10 molar % (e.g., from about 1.5 molar % to about 9.5 molar %, from about 2 molar % to about 9 molar %, from about 2.5 molar % to about 8.5 molar %, from about 3 molar % to about 8 molar %, from about 3.5 molar % to about 7.5 molar %, from about 4 molar % to about 7 molar %, from about 4.5 molar % to about 6.5 molar %, from about 5 molar % to about 6 molar %, from about 1 molar % to about 5.5. molar %, from about 1.5 molar % to about 5 molar %, from about 2 molar % to about 4.5 molar %, from about 2.5 molar % to about 4 molar %, from about 3 molar % to about 3.5 molar %, from about 5.5. molar % to about 10 molar %, from about 6 molar % to about 9.5 molar %, from about 6.5 molar % to about 9 molar %, from about 7 molar % to about 8.5 molar %, from about 7.5 molar % to about 8 molar %) of the PEGylated lipid.

In some aspects, the leukocyte membrane protein can be derived from a leukocyte plasma membrane. In some such aspects, the leukocyte plasma membrane can be derived from a human leukocyte plasma membrane. In other such aspects, the leukocyte plasma membrane can be derived from a murine leukocyte plasma membrane. In other aspects, the leukocyte membrane protein can be a synthetic recombinant protein.

In some aspects, the leukocyte membrane protein can be lymphocyte function-associated antigen 1 (LFA-1), CD11, CD45, CD47, or any combination thereof. In some aspects, the leukocyte membrane protein can include some or all of the peptides present in a leukocyte plasma membrane (e.g., a human leukocyte plasma membrane or a murine leukocyte plasma membrane).

In some aspects, the biomimetic proteolipid nanovesicle can further include an N/P ratio of at least about 3 (e.g., at least about 3.25, at least about 3.5, at least about 3.75, at least about 4, at least about 4.25, at least about 4.5, at least about 4.75, at least about 5, at least about 5.25, at least about 5.5, at least about 5.75, at least about 6, at least about 6.25, at least about 6.5, at least about 6.75, at least about 7, at least about 7.25, at least about 7.5, at least about 7.75, at least about 8, at least about 8.25, at least about 8.5, at least about 8.75, at least about 9, at least about 9.25, at least about 9.5, at least about 9.75, at least about 10). In some aspects, the biomimetic proteolipid nanovesicle can further include an N/P ratio of up to about 10 (e.g., up to about 9.75, up to about 9.5, up to about 9.25, up to about 9, up to about 8.75, up to about 8.5, up to about 8.25, up to about 8, up to about 7.75, up to about 7.5, up to about 7.25, up to about 7, up to about 6.75, up to about 6.5, up to about 6.25, up to about 6, up to about 5.75, up to about 5.5, up to about 5.25, up to about 5, up to about 4.75, up to about 4.5, up to about 4.25, up to about 4, up to about 3.75, up to about 3.5, up to about 3.25, up to about 3). In some aspects, the biomimetic proteolipid nanovesicle can further include an N/P ratio of about 3, about 3.25, about 3.5, about 3.75, about 4, about 4.25, about 4.5, about 4.75, about 5, about 5.25, about 5.5, about 5.75, about 6, about 6.25, about 6.5, about 6.75, about 7, about 7.25, about 7.5, about 7.75, about 8, about 8.25, about 8.5, about 8.75, about 9, about 9.25, about 9.5, about 9.75, or about 10.

It is considered that the biomimetic proteolipid nanovesicle can have an N/P ratio ranging from any of the minimum values described above to any of the maximum values described above. For example, in some aspects, the biomimetic proteolipid nanovesicle can have an N/P ratio of from about 3 to about 10 (e.g., from about 3.25 to about 9.75, from about 3.5 to about 9.5, from about 3.75 to about 9.25, from about 4 to about 9, from about 4.25 to about 8.75, from about 4.5 to about 8.5, from about 4.75 to about 8.25, from about 5 to about 8, from about 5.25 to about 7.75, from about 5.5 to about 7.5, form about 5.75 to about 7.25, from about 6 to about 7, from about 6.25 to about 6.75, from about 3 to about 6.5, from about 3.25 to about 6.25, from about 3.5 to about 6, from about 3.75 to about 5.75, from about 4 to about 5.5, from about 4.25 to about 5.25, from about 4.5 to about 5, from about 6.5 to about 10, from about 6.75 to about 9.75, from about 7 to about 9.5, from about 7.25 to about 9.25, from about 7.5 to about 9, from about 7.75 to about 8.75, from about 8 to about 8.5).

In some aspects, the biomimetic proteolipid nanovesicle can have a diameter of at least about 50 nm (e.g., at least about 60 nm, at least about 70 nm, at least about 80 nm, at least about 90 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm). In some aspects, the biomimetic proteolipid nanovesicle can have a diameter of up to about 200 nm (e.g., up to about 190 nm, up to about 180 nm, up to about 170 nm, up to about 160 nm, up to about 150 nm, up to about 140 nm, up to about 130 nm, up to about 120 nm, up to about 110 nm, up to about 100 nm, up to about 90 nm, up to about 80 nm, up to about 70 nm, up to about 60 nm, up to about 50 nm). In some aspects, the biomimetic proteolipid nanovesicle can have a diameter of about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, or about 200 nm.

It is considered that the biomimetic proteolipid nanovesicle can have a diameter ranging from any of the minimum values described above to any of the maximum values described above. For example, in some aspects, the biomimetic proteolipid nanovesicle can have a diameter of from about 50 nm to about 200 nm (e.g., from about 60 nm to about 190 nm, from about 70 nm to about 180 nm, from about 80 nm to about 170 nm, from about 90 nm to about 160 nm, from about 100 nm to about 150 nm, from about 110 nm to about 140 nm, from about 120 nm to about 130 nm, from about 50 nm to about 130 nm, from about 60 nm to about 120 nm, from about 70 nm to about 110 nm, from about 80 nm to about 100 nm, from about 120 nm to about 200 nm, from about 130 nm to about 190 nm, from about 140 nm to about 180 nm, from about 150 nm to about 170 nm).

In some aspects, the mRNA can be targeted to treat a cancer, inflammation, an infectious disease, or a genetic disease or disorder.

### Methods

In an aspect, provided is a method of making any of the disclosed biomimetic proteolipid nanovesicles, the method including: a) dissolving a phosphocholine-based phospholipid, an ionizable or cationic lipid, and a cholesterol in an organic solvent to produce an organic lipid solution; b) dissolving aleukocyte membrane protein and an agent in water to produce an aqueous protein solution; and c) loading the organic lipid solution into an organic phase inlet of a microfluidic mixer, and loading the aqueous protein solution into an aqueous phase inlet of said microfluidic mixer; and d) adjusting flow rates of each inlet stream and a flow ratio between each inlet stream to produce the biomimetic proteolipid nanovesicles having a specified lipid-to-protein ratio by weight therefrom.

In some aspects, the agent can include a nucleic acid. In some such aspects, the agent can include an siRNA and/or an mRNA.

In some aspects (e.g., when the agent is an siRNA), the method can produce biomimetic proteolipid nanovesicles having a lipid-to-protein ratio of at least about 1:50 (e.g., at least about 1:55, at least about 1:60, at least about 1:65, at least about 1:66, at least about 1:67, at least about 1:68, at least about 1:69, at least about 1:70, at least about 1:71, at least about 1:72, at least about 1:73, at least about 1:74, at least about 1:75, at least about 1:76, at least about 1:77, at least about 1:78, at least about 1:79, at least about 1:80, at least about 1:81, at least about 1:82, at least about 1:83, at least about 1:84, at least about 1:85, at least about 1:90, at least about 1:95, at least about 1:100) by weight. In some aspects, the method can produce biomimetic proteolipid nanovesicles having a lipid-to-protein ratio of up to about 1:100 (e.g., up to about 1:95, up to about 1:90, up to about 1:85, up to about 1:84, up to about 1:83, up to about 1:82, up to about 1:81, up to about 1:80, up to about 1:79, up to about 1:78, up to about 1:77, up to about 1:76, up to about 1:75, up to about 1:74, up to about 1:73, up to about 1:72, up to about 1:71, up to about 1:70, up to about 1:69, up to about 1:68, up to about 1:67, up to about 1:66, up to about 1:65) by weight. In some aspects, the method can produce biomimetic proteolipid nanovesicles having a lipid-to-protein ratio of about 1:50, 1:55, 1:60, 1:65, about 1:66, about 1:67, about 1:68, about 1:69, about 1:70, about 1:71, about 1:72, about 1:73, about 1:74, about 1:75, about 1:76, about 1:77, about 1:78, about 1:79, about 1:80, about 1:81, about 1:82, about 1:83, about 1:84, about 1:85, about 1:90, about 1:95, or about 1:100 by weight.

It is considered that the method can produce biomimetic proteolipid nanovesicles having a lipid-to-protein ratio ranging from any of the minimum values described above to any of the maximum values described above. For example, in some aspects (e.g., when the agent is an siRNA), the method can produce biomimetic proteolipid nanovesicles having a lipid-to-protein ratio of from about 1:65 to about 1:85 (e.g., from about 1:66 to about 1:84, from about 1:67 to about 1:83, from about 1:68 to about 1:82, from about 1:69 to about 1:81, from about 1:70 to about 1:80, from about 1:71 to about 1:79, from about 1:72 to about 1:78, from about 1:73 to about 1:77, from about 1:74 to about 1:76, from about 1:65 to about 1:75, from about 1:66 to about 1:74, from about 1:67 to about 1:73, from about 1:68 to about 1:72, from about 1:69 to about 1:71, from about 1:75 to about 1:85, from about 1:76 to about 1:84, from about 1:77 to about 1:83, from about 1:78 to about 1:82, from about 1:79 to about 1:81) by weight. In some aspects, (e.g., when the agent is an siRNA), the method can produce biomimetic proteolipid nanovesicles having a lipid-to-protein ratio of from about 1:50 to about 1:100 by weight.

In some aspects (e.g., when the agent is an mRNA), the method can produce biomimetic proteolipid nanovesicles having a lipid-to-protein ratio of at least about 1:80 (e.g., at least about at least about 1:81, at least about 1:82, at least about 1:83, at least about 1:84, at least about 1:85, at least about 1:86, at least about 1:87, at least about 1:88, at least about 1:89, at least about 1:90, at least about 1:91, at least about 1:92, at least about 1:93, at least about 1:94, at least about 1:95, at least about 1:96, at least about 1:97, at least about 1:98, at least about 1:99, at least about 1:100) by weight. In some aspects, the method can produce biomimetic proteolipid nanovesicles having a lipid-to-protein ratio of up to about 1:100 (e.g., up to about 1:99, up to about 1:98, up to about 1:97, up to about 1:96, up to about 1:95, up to about 1:94, up to about 1:93, up to about 1:92, up to about 1:91, up to about 1:90, up to about 1:89, up to about 1:88, up to about 1:87, up to about 1:86, up to about 1:85, up to about 1:84, up to about 1:83, up to about 1:82, up to about 1:81, up to about 1:80) by weight. In some aspects, the method can produce biomimetic proteolipid nanovesicles having a lipid-to-protein ratio of about 1:80, about 1:81, about 1:82, about 1:83, about 1:84, about 1:85, about 1:86, about 1:87, about 1:88, about 1:89, about 1:90, about 1:91, about 1:92, about 1:93, about 1:94, about 1:95, about 1:96, about 1:97, about 1:98, about 1:99, or about 1:100 by weight.

It is considered that the method can produce biomimetic proteolipid nanovesicles having a lipid-to-protein ratio ranging from any of the minimum values described above to any of the maximum values described above. For example, in some aspects (e.g., when the agent is an mRNA), the method can produce biomimetic proteolipid nanovesicles having a lipid-to-protein ratio of from about 1:80 to about 1:100 (e.g., from about 1:81 to about 1:99, from about 1:82 to about 1:98, from about 1:83 to about 1:97, from about 1:84 to about 1:96, from about 1:85 to about 1:95, from about 1:86 to about 1:94, from about 1:87 to about 1:93, from about 1:88 to about 1:92, from about 1:89 to about 1:91, from about 1:80 to about 1:90, from about 1:81 to about 1:89, from about 1:82 to about 1:88, from about 1:83 to about 1:87, from about 1:84 to about 1:86, from about 1:90 to about 1:100, from about 1:91 to about 1:99, from about 1:92 to about 1:98, from about 1:93 to about 1:97, from about 1:94 to about 1:96) by weight.

In some aspects, the total combined flow rate of both inlet streams can be at least about 2 mL/min (e.g., at least about 3 mL/min, at least about 4 mL/min, at least about 5 mL/min, at least about 6 mL/min, at least about 7 mL/min, at least about 8 mL/min, at least about 9 mL/min, at least about 10 m/min, at least about 11 ml/min, at least about 12 mL/min, at least about 13 mL/min, at least about 14 mL/min, at least about 15 mL/min). In some aspects, the total combined flow rate of both inlet streams can be up to about 15 mL/min (e.g., up to about 14 mL/min, up to about 13 mL/min, up to about 12 mL/min, up to about 11 mL/min, up to about 10 mL/min, up to about 9 mL/min, up to about 8 mL/min, up to about 7 mL/min, up to about 6 m/min, up to about 5 mL/min, Lip to about 4 mL/min, up to about 3 mL/min, up to about 2 mL/min). In some aspects, the total combined flow rate of both inlet streams can be about 2 mL/min, about 3 mL/min, about mL/min, about e mL/min, about 6 mL/min, about 7 mL/min, about 8 mL/min, about 9 mL/min, about 10 mL/min, about 11 m/min, about 12 m/min, about 13 m/min, about 14 mL/min, or about 15 mL/min.

It is considered that the total combined flow rate of both inlet streams can range from any of the minimum values described above to any of the maximum values described above. For example, the total combined flow rate of both inlet streams can be from about 2 mL/min to about 15 mL/min (e.g., from about 3 mL/min to about 14 mL/min, from about 4 mL/min to about 13 mL/min, from about 5 mL/min to about 12 mL/min, from about 6 L/min to about 11 mL/min, from about 7 mL/min to about 10 mL/min, from about 8 mL/min to about 9 mL/min, from about 2 mL/min to about 9 mL/min, from about 3 mL/min to about 8 mL/min, from about 4 mL/min to about 7 ml/mi, from about 5 mL/min to about 6 mL/min, from about 8 mL/min to about 15 mL/min, from about 9 ml/min to about 14 mL/min, from about 10 mL/min to about 13 mL/min, from about 11 mL/min to about 12 mL/min).

In some aspects, the flow ratio between the organic phase inlet and the aqueous phase inlet can be at least about 1:2 (e.g., at least about 1:2.5, at least about 1:3, at least about 1:3.5, at least about 1:4, at least about 1:4.5, at least about 1:5). In some aspects, the flow ratio between the organic phase inlet and the aqueous phase inlet can be up to about 1:5 (e.g., up to about 1:4.5, up to about 1:4, up to about 1:3.5, up to about 1:3, up to about 1:2.5, up to about 1:2.). In some aspects, the flow ratio between the organic phase inlet and the aqueous phase inlet can be about 1:2, about 1:2,5, about 1:3, about 1:3.5, about 1:4, about 1:4.5, or about 1:5.

It is considered that the flow ratio between the organic phase inlet and the aqueous phase inlet can range from any of the minimum values described above to any of the maximum values described above. For example, in some aspects, the flow ratio between the organic phase inlet and the aqueous phase inlet can be from about 1:2 to about 1:5 (e.g., from about 1:2.5 to about 1:4.5, from about 1:3 to about 1:4, from about 1:2 to about 1:3.5, from about 1:2.5 to about 1:3, from about 1:3.5 to about 1:5, from about 1:4 to about 1:4.5)

In some aspects, the aqueous protein solution can have a protein concentration of at least about 2 mg/ml (e.g., at least about 2.5 mg/mL, at least about 3 mg/mL, at least about 3.5 mg/mL, at least about 4 mg/mL, at least about 4.5 mg/mL, at least about 5 mg/mL, at least about 5.5 mg/mL, at least about 6 mg/mL, at least about 6.5 mg/mL, at least about 7 mg/mL, at least about 7.5 mg/mL, at least about 8 mg/mL). In some aspects, the aqueous protein solution can have a protein concentration of up to about 8 mg/mL (e.g., up to about 7.5 mg/mL, up to about 7 mg/mL, up to about 6.5 mg/mL, up to about 6 mg/mL, up to about 5.5 mg/mL, up to about 5 mg/mL, up to about 4.5 mg/mL, up to about 4 mg/mL, up to about 3.5 mg/mL, up to about 3 mg/mL, up to about 2.5 mg/mL, up to about 2 mg/mL). In some aspects, the aqueous protein solution can have a protein concentration of about 2 mg/mL, about 2.5 mg/mL, about 3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, or about 8 mg/mL.

It is considered that the aqueous protein solution can have a protein concentration ranging from any of the minimum values described above to any of the maximum values described above. For example, in some aspects, the aqueous protein solution can have a protein concentration of from about 2 mg/mL to about 8 mg/mL (e.g., from about 2.5 mg/mL, from about 7.5 mg/mL, from about 3 mg/mL to about 7 mg/mL, from about 3.5 mg/mL to about 6.5 mg/mL, from about 4 mg/mL to about 6 mg/mL, from about 4.5 mg/mL to about 5.5 mg/mL, from about 2 mg/mL to about 5 mg/mL, from about 2.5 mg/mL to about 4.5 mg/mL, from about 3 mg/mL to about 4 mg/mL, from about 5 mg/mL to about 8 mg/mL, from about 5.5 mg/mL to about 7.5 mg/mL, from about 6 mg/mL to about 7 mg/mL).

In some aspects, the method can yield at least about 60% (e.g., at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%) agent encapsulation efficiency.

In some aspects, step c) can be performed at a temperature of at least about 40° C. (e.g., at least about 41° C., at least about 42° C.′, at least about 43° C., at least about 44° C., at least about 45° C., at least about 46° C., at least about 47° C., at least about 48° C., at least about 49° C., at least about 50° C.). In some aspects, step c) can be performed at a temperature of up to about 50° C. (e.g., up to about 49° C., up to about 48° C., up to about 47° C., up to about 46° C., up to about 45° C., up to about 44° C., up to about 43° C., up to about 42° C., up to about 41° C., up to about 40° C.). In some aspects, step c) can be performed at a temperature of about 40° C., about 41° C., about 42° C., about 43° C., about 44° C., about 45° C., about 46° C., about 47° C., about 48° C., about 49° C., or about 50° C.

It is considered that step c) can be performed at a temperature ranging from any of the minimum values described above to any of the maximum values described above. For example, in some aspects, step c) can be performed at a temperature of from about 40° C. to about 50° C. (e.g., from about 41° C. to about 49° C., from about 42° C. to about 48° C., from about 43° C. to about 47° C., from about 44° C. to about 46° C., from about 40° C.′ to about 45° C., from about 41° C. to about 44° C., from about 42° C. to about 43° C., from about 45° C. to about 50° C., from about 46° C. to about 49° C., from about 47° C. to about 48° C.).

In another aspect, provided is a method for the delivery of an agent into a cell, the method including introducing into the cell any of the disclosed biomimetic proteolipid nanovesicles.

In some aspects, the agent can include a nucleic acid. In some such aspects, the agent can include an siRNA and/or an mRNA.

In some aspects, the method can be used to target delivery of the agent to inflamed tissues.

In some aspects, the method can be performed in vivo, in vitro, or ex vivo.

In some aspects, the cell can be human.

In yet another aspect, provided is a method of treating a disease or disorder in a subject in need thereof, the method including administering to the subject any of the disclosed biomimetic proteolipid nanovesicles.

In some aspects, the agent can include a nucleic acid. In some such aspects, the agent can include an siRNA and/or an mRNA.

In some aspects, the disease or disorder can be a cancer. In some such aspects, the disease or disorder can be triple negative breast cancer. Additionally or alternatively, in other aspects, the disease or disorder can be inflammation. In some such aspects, the disease or disorder can be a tumor, sepsis, a traumatic brain injury, inflamed epithelia, atherosclerosis, or post traumatic osteoarthritis.

### Examples

### Example 1: siRNA Loading into Leukosomes

In addition to the standard-of-care utilized in the treatment of TNBC, targeted delivery of RNA represents a promising alternative approach. Given the various signaling pathways known to act as drivers of the aggressiveness of this disease, modulation of these dysregulated pathways can be achieved through RNA molecules. In particular, siRNA can serve as molecular brakes that reduce the expression of proteins activating signaling within these pathways^{[1, 2]}. However, administration of these molecules requires the use of a protective carrier that takes these molecules to the target site^{[3]}. The harsh in vivo environment exposes the siRNA to enzymes that quickly degrade the molecules upon systemic administration^{[4]}. As a result, NPs have been utilized as protective drug delivery carriers of siRNA^{[5]}.

To date, NP-based siRNA delivery has been tested using polymeric, lipid and inorganic NPs^{[4,6]}. Most notably, lipid NPs have demonstrated the most promising potential as carriers of siRNA^{[7]}. Recent FDA approval of two lipid-based NP-siRNA formulations has validated the use of these NPs for the treatment of liver diseases^{[8]}. This has been primarily due to the natural homing properties of these lipidic formulations to the liver^{[9-11]}. However, researchers have explored ways to further the utility of these NPs for other organs, which has required iterative testing of formulations for organ-specific targeting^{[12]}. Attempts to address this current gap in the application of this technology to other diseases have included synthesis of novel lipids and tuning the lipid contents of the NP formulation^{[11, 14]}. However, no studies exist to date utilizing a protein-based approach for organ-specific targeting of these NPs.

Building off the previous study corroborating the ability of the leukosomes to effectively target the TNBC tumor, this work aimed to explore if the leukosome formulation can be applied to RNA delivery. First, several lipid backbones were screened to identify which would be most amenable for siRNA loading. Then, the selected lipid backbone was tested for its ability to integrate leukocyte membrane proteins while maintaining desired NP physiochemical properties. Upon identifying the need to further investigate the formulation, effects of tuning the lipid composition and synthesis parameters were studied. Following these studies, two formulations were selected for further in vitro and in vivo analysis. In vitro studies were comprised of cytotoxicity and gene expression analysis, while a TNBC tumor model was utilized to study the NP biodistribution. Having identified the JAK/STAT3 signaling axis to be overactive in TNBC and associated with poor prognosis in TNBC patients, STAT3 siRNA was selected as the model molecule to demonstrate the delivery capabilities of this novel formulation.

### Materials and Methods

Cell culture: J774. A1 (murine macrophages), 4T1 (murine TNBC) and MDA-MB-231-GFP (human TNBC) cells were cultured in DMEM HG media with 10% fetal bovine serum, 1% pen-strep and 1% L-glutamine.

Protein extraction for NPs synthesis: Protein extraction of the membrane proteins from J774 cells was performed using a Calbiochem ProteoExtract Kit. Approximately 50 million cells were split evenly into 10 tubes (5 million cells/tube), washed twice with provided Wash Buffer and centrifuged at 200 g for 10 min at 4° C. following each wash. The supernatant was removed, pellet resuspended in 2 mL of extraction buffer 1 and incubated on ice for 10 min under gentle agitation. Samples were then centrifuged at 16000 g for 15 min at 4° C., followed by removal of the supernatant. Next, the pellet was resuspended in 1 mL of extraction buffer 2 (EB2) and incubated on ice for 30 min under gentle agitation. Samples were centrifuged once again at 16000 g for 15 min at 4° C. Lastly, the supernatant containing the extracted membrane proteins was transferred to a fresh tube and stored at −80° C.

siRNA: Scramble siRNA, also known as Negative Control siRNA #1, was purchased from Ambion. STAT3 siRNA for murine cells was purchased from Qiagen, while GFP siRNA purchased from Ambion.

Synthesis of liposomes and leukosomes with siRNA: Initial screening for the selection of the lipid backbones involved synthesis of 3 different lipid formulations. 3 lipid backbones were tested with the cationic or ionizable lipid being different in each one. DOTAP was tested a cationic lipid for siRNA loading, while 16:0 DAP and DLin-MC3-DMA were tested as ionizable lipids of choice. Two phases (i.e. aqueous and organic) was utilized in the synthesis of the NPs on the NanoAssemblr™ system. Lipids and ethanol made up the organic phase, while acidic sodium acetate buffer, siRNA and protein or protein buffer comprised the aqueous phase. Following synthesis on the NanoAssemblr™, NPs were in dialyzed overnight in a two-step process utilizing acidic sodium acetate buffer to remove residual ethanol followed by 1×PBS. All NPs were filtered using a 0.22 μM PVDF filters prior to characterization and treatment of cells or mice. Fluorescently labeled siRNA-NPs were synthesized using the addition of a rhodamine-lipid during the synthesis process, with no changes to the parameters on the NanoAssemblr™ or post-synthesis purification methods.

Dynamic light scattering, zeta potential and concentration of NPs: Malvern Zetasizer was utilized to measure the size, polydispersity index (PDI) and zeta potential of the NPs. For size and PDI measurements, samples were diluted 1:1000 in MilliQ water and characterized with 3 measurements of 15 runs for each. Final values for each sample were calculated as the average of the 3 measurements. Samples for zeta potential characterization were prepared by diluting NPs 1:1000 in MilliQ water and PBS (1:10).

RNA loading quantification: A modified Ribogreen© assay was utilized for the quantification of siRNA loaded within the synthesized NPs, as previously described^{[15]}. NP samples were first diluted 1:100 in 1×TE buffer, followed by 50 μL of each diluted sample added to either 50 μL of 1×TE buffer or 2% Triton-X 100 in 1×TE buffer. RNA standard curve, ranging from 0.1-2.5 μg/mL, was prepared with using a combination of 2% Triton-X 100 in 1×TE buffer and 1×TE buffer as the diluent (TABLE 1). All samples were run in duplicate and incubated at 37 C for 10 minutes. Ribogreen reagent was diluted 1:100 in 1×TE buffer, with 100 uL added to each well. Following a 4 min incubation at room temperature, plate was read at Ex/Em of 480 nm/520 nm.

TABLE 1
Sample preparation of RNA standards for Ribogreen ™ kit

| RNA | RNA | TE | Triton | Total |
|---|---|---|---|---|
| concentration | stock | buffer | buffer | volume per |
| (μg/mL) | (μL) | (μL) | (μL) | well (μL) |
| 2.5 | 25 | 25 | 50 | 100 |
| 1 | 10 | 40 | 50 | 100 |
| 0.5 | 5 | 45 | 50 | 100 |
| 0.25 | 2.5 | 47.5 | 50 | 100 |
| 0.1 | 1 | 49 | 50 | 100 |

Cytotoxicity of leukosomes: 4T1 cells were seeded in complete media in a 96-well plate at a seeding density of 8,000 cells/well. After 24 h, NP were resuspended in Optimem media and added to the cells at the following concentrations: 10, 25, 50, 100, 250, 500 and 1000 μM. These concentrations were based on the lipid concentration of the NP after synthesis. Following 6 h of incubation with NPs, Optimem media was aspirated and replaced with complete DMEM HG media. Toxicity of NP treatment was evaluated using an MTT assay at 3 timepoints—24, 48 and 72 h. At each timepoint, media was aspirated and replaced with MTT resuspended in completed media at a concentration of 0.5 mg/ml. After 2 h, MTT reagent was aspirated and replaced with equal volume DMSO. Following 30 minutes of gentle agitation at room temperature, absorbance was measured at 570 nm with reference wavelength of 630 nm.

Gene expression analysis for knockdown efficiency: 4T1 cells were seeded in complete media in a 24-well plate at a seeding density of 45,000 cells/well. After 24 h, NP were resuspended in Optimem media and added to the cells. Following 6 h of incubation with NPs, Optimem media was aspirated and replaced with complete DMEM HG media. Gene expression analysis was performed at 3 timepoints—24, 48 and 72 h. At each timepoint, cells were washed twice with 1×PBS followed by the addition of 250 μL of lysis buffer added to each well. RNA was extracted using a Qiagen RNeasy kit followed by RNA quantification using a NanoDrop. cDNA samples were prepared using an iScript cDNA synthesis kit followed by synthesis on a Biorad CFX system.

Imaging for GFP expression knockdown: GFP-expression MDA-MB-231 cells were seeded in complete media in a 96-well plate at a seeding density of 8,000 cells/well. After 24 h, NP were resuspended in Optimem media and added to the cells. Following 6 h of incubation with NPs, Optimem media was aspirated and replaced with complete DMEM HG media. Treated plate was then placed onto an Incucyte imaging system for interval imaging for 72 h using a 10× objective.

In vivo model of TNBC: All animal experiments were performed in accordance with the guidelines of the Animal Welfare Act and the Guide for the Care and Use of Laboratory Animals approved by The Houston Methodist Institutional Animal Care and Use Committee guidelines (Houston, TX). TNBC model was established by injecting a total of 3×10⁵ 4T1-Red-FLuc (PerkinElmer, Waltham MA) cells, suspended in 50 μl of 1×PBS, subcutaneously into the mammary fat pad of 10-week-old BALB/c female mice (Charles River Laboratories, Wilmington, MA). 10-12 days after tumor inoculation, mice were divided into 3 groups—untreated control, siRNA-liposomes and siRNA-leukosomes. At each timepoint, mice were euthanized and organs collected for imaging on IVIS® Spectrum. Image acquisition parameters were the following: excitation=535 nm, emission=600 nm, f stop=2, binning=medium.

Recognizing the need to tune the lipid formulation in order to facilitate the integration of leukocyte membrane proteins while maintaining desired NP physiochemical properties, initial tests focused on understanding the effects of interchanging lipids present in the original lipid backbone.

Previous studies have demonstrated the antifouling properties of PEG and, thereby, exhibiting protein-repellent properties^{[16]}. Therefore, this work tested the hypothesis if the presence of PEG interfered with the integration of membrane proteins and resulted in the previously observed higher size and PDI of the NPs. However, removal of the PEG lipid in the DLin-MC3 NPs resulted in a significant increase in both the size and PDI of liposomes and leukosomes (FIGS. 28A-28B). Liposomes without the PEG lipid were 2-fold greater in size, while leukosomes were 1.5-fold higher (FIG. 28A). Absence of the PEG lipid had even more profound effects on the PDI of the leukosomes, with almost a 200% increase in the PDI (FIG. 28B). As previously observed, siRNA-leukosomes with PEG had more negative surface charge than siRNA-liposomes with PEG (FIG. 28C). Removal of the PEG reduced this negative charge and making the siRNA-leukosomes surface charge similar to that of the siRNA-liposomes with PEG. Therefore, these results contradicted our initial hypothesis and supported the notion that the presence of PEG was in fact vital for the assembly and stability of the synthesized NPs, both those with and without protein.

### Results

Identification and validation of lipid backbone for siRNA loading: Development of a leukosome formulation amenable for siRNA required identification of a lipid backbone that would allow for loading of RNA and integration of the extracted membrane proteins. Previous studies have demonstrated the need for positively charged components in a nanoparticle system in order to effectively load negatively charged RNA molecules. To this end, 3 lipid backbones were screened for their ability to effectively load siRNA, maintain key NP physiochemical properties and exhibit in vitro knockdown efficiencies with minimal cytotoxicity to TNBC cells.

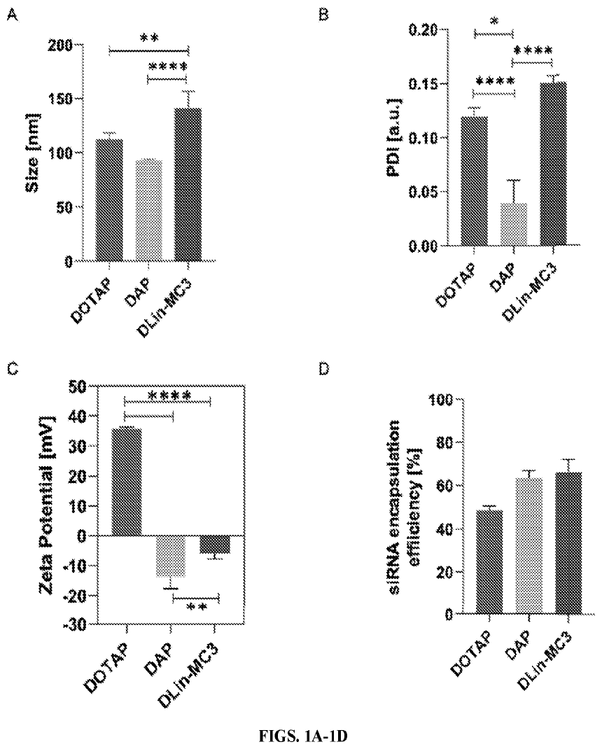

Liposomes comprised of 3 discrete lipid formulations and loaded with scramble siRNA were synthesized on the NanoAssemblr and characterized for their physiochemical properties. All 3 lipid formulations maintained a size below 200 nm and a PDI less than 0.2 (FIGS. 1A-1B). However, significant differences could be observed in the zeta potential of the 3 formulations, with DOTAP lipid nanoparticles having a positive charge following synthesis and dialysis processing (FIG. 1C). In addition, DOTAP exhibited the lowest siRNA encapsulation efficiency among the 3 lipid backbones screened, with only 50% of siRNA encapsulated within the NPs (FIG. 1D).

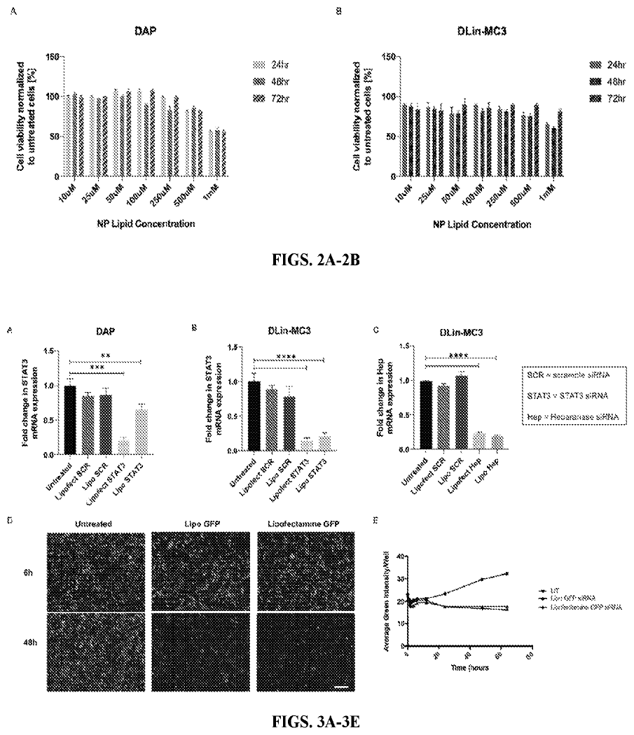

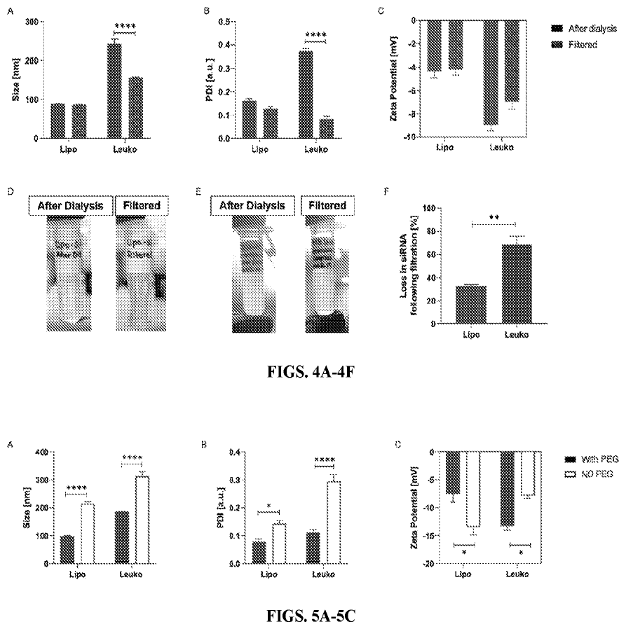

Prior to evaluating the in vitro knockdown efficiency of the 2 selected formulations, cytotoxic profile of each liposome formulation was evaluated on 4T1 cells (FIGS. 2A-2B). In both formulations, analysis indicated minimal decreases in viability for up to 72 h at concentrations less than 500 μM. Following establishment of the max NP concentration threshold, siRNA knockdown efficiency of both formulations was determined. Treatment of 4T1 cells with DAP NPs loaded with STAT3-siRNA resulted in only a 40% knockdown of STAT3 mRNA levels after 24 h (FIG. 3A). This was knockdown efficiency was only 50% of that achieved by the Lipofectamine control which exhibited an 80% knockdown in STAT3 expression (FIG. 3A). On the other hand, DLin-MC3 liposomes loaded with the same STAT3-siRNA demonstrated an 80% in knockdown efficiency, with performance comparable to the Lipofectamine control (FIG. 3B). Further evaluation of the knockdown efficiency of DLin-MC3 liposomes corroborated the robust abilities of this lipid backbone for siRNA delivery. Loading of a different siRNA molecule (i.e. Heparanase-siRNA) resulted in the same knockdown efficiency as that observed with STAT3-siRNA NPs (FIG. 3C). In addition, the superior knockdown efficacy of this formulation was found to be not limited to cellular mRNA levels alone. Treatment of GFP-expressing TNBC cells with GFP-siRNA loaded DLin-MC3 liposomes resulted in a 75% decrease in detected green signal, indicative of a decrease in the production of the GFP protein (FIGS. 3D-3E). This observed cellular in response in GFP protein production validated the ability of the delivered siRNA to reduce both mRNA and protein levels of the target protein.

Following this lipid screen, NPs made utilizing the DLin-MC3 formulation were determined to be the superior candidate for moving forward with the leukosome formulation.

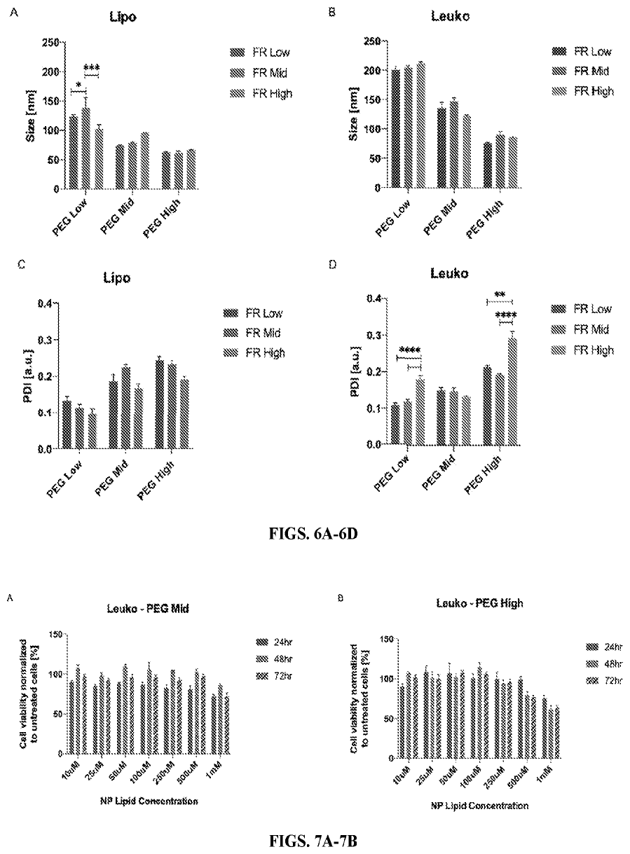

Effect of tuning lipid composition on siRNA-leukosomes' physiochemical properties: Having identified the DLin-MC3 lipid backbone as the most promising candidate for siRNA-loading, synthesis of siRNA-leukosomes integrating proteins into these lipid NPs was tested. Following synthesis of these NPs, a significant difference could be observed not only between the liposomes and the leukosomes, but also in the NP properties following filtration. siRNA-leukosomes collected after dialysis were over 200 nm with a large PDI of 0.4 (FIGS. 4A-4B). In addition, there was 50 nm difference between filtered liposomes and leukosomes, a difference that could have significant effects when comparing the in vivo targeting behavior of these NPs. While both liposomes and leukosomes maintained an overall negative surface charge (FIG. 4C), visual inspection of the NPs both after dialysis and filtration confirmed the size and PDI data acquired (FIG. 4D). In particular, siRNA-leukosomes collected after dialysis were opaquer, with solutions becoming clearer following filtration (FIG. 4D). In contrast, siRNA-liposomes were clear both after dialysis and filtration (FIG. 4E). Recognizing this change in solution transparency must also be associated with a loss of material in the filtration process, siRNA encapsulation was quantified both before and after the filtration process. Indeed, filtration of the siRNA-leukosomes resulted in almost 70% loss of siRNA, while liposomes only had about a 30% in loss (FIG. 4F).

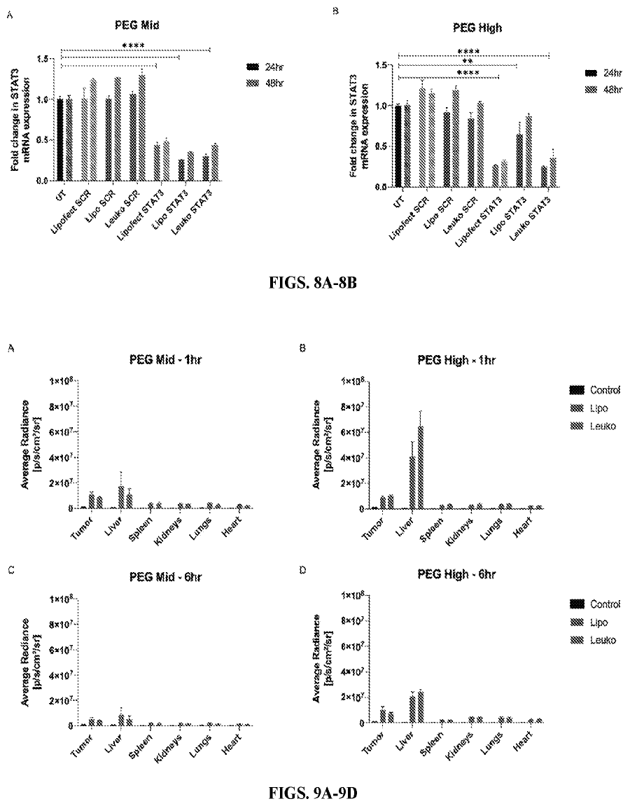

Upon understanding the role of the PEG lipid in stabilizing the siRNA-leukosomes, efforts were shifted towards understanding how the PEG concentration can be tuned to further stabilize the leukosomes. To this end, NPs were synthesized using 2-fold and 4-fold more PEG lipid than the original lipid backbone with the lowest PEG content. Evaluation of the resulting NPs demonstrated the packing effect of increasing the PEG amount on the NPs. Increasing the percentage of PEG lipid caused a 50% decrease in the size of liposomes, with no changes observed when increasing from mid to high percentage of PEG (FIG. 5A). The effects of this tuning were more apparent in the leukosomes, with almost a 50% reduction in the size when using the highest percentage of PEG (FIG. 5A). On the other hand, NPs with increasing PEG content demonstrated an incremental increase in the PDI, with both Lipo- and Leuko-PEG 6% having a PDI between 0.2-0.25 (FIG. 5B). siRNA encapsulation efficiency in both siRNA-liposomes and leukosomes was found to increase by 20% when increasing the PEG content (FIG. 5C). Notably, evaluation of siRNA loss during the filtration process was found to significantly decrease with increasing PEG formulations of both liposomes and leukosomes, with less than 20% loss in the highest PEG NPs (FIG. 5D). Taken together, these results indicated that increasing the PEG concentration has significant effects in stabilizing the siRNA leukosomes.

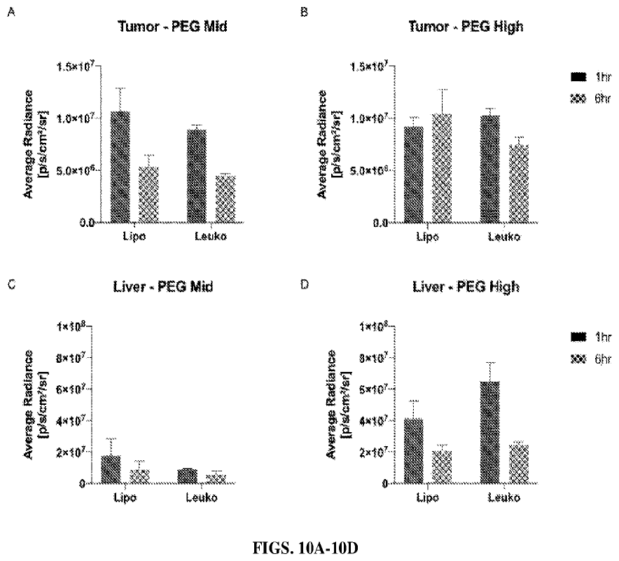

Effect of synthesis parameters on siRNA-leukosomes' physiochemical properties: In addition to the lipid composition of the NPs, synthesis parameters served as the other component of the formulation that can be tuned to affect the subsequent NP properties. In particular, the flow rate (FR) utilized during the synthesis on the NanoAssemblr™ was hypothesized to have an effect on the resulting NPs^{[17]}. However, synthesis of siRNA-leukosomes with varying concentrations of PEG at a low, mid and high flow rates was found to have minimal effects on the size and PDI of the NPs (FIGS. 6A-6D). Notably, the synthesis of the leukosomes at the highest flow rate Given that both the mid and high PEG percentage NPs demonstrated stabilization of the NPs, especially in terms of size and siRNA encapsulation, both of these of PEG ratios were selected to continue to in vitro and in vivo evaluation. Both of these formulations were synthesized using the mid flow rate.

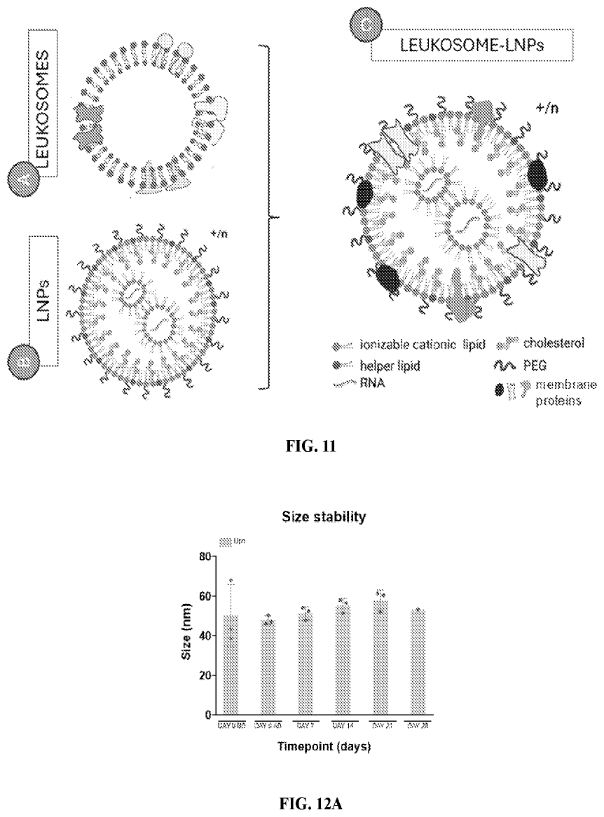

Cytotoxicity profile of 2 candidate siRNA-leukosomes formulations: Prior to validating the knockdown efficiency of siRNA-loaded NPs, the 2 selected siRNA-leukosome formulations were evaluated for their cytotoxic profile on 4T1 cells. siRNA leukosomes with the mid PEG percentage exhibited minimal decreases in cell viability, with cells demonstrating a recovery in viability after the first 24 h (FIG. 7A). Treatment with leukosomes of the highest concentration of PEG resulted in minimum decrease in cell viability for 72 h at concentrations below 250 μM (FIG. 7B). Both concentrations above this threshold resulted in a 40% decrease in cell viability 72 h after NP treatment (FIG. 7B). Taken together, 250 μM was determined as the maximum threshold of treatment for subsequent in vitro experiments.

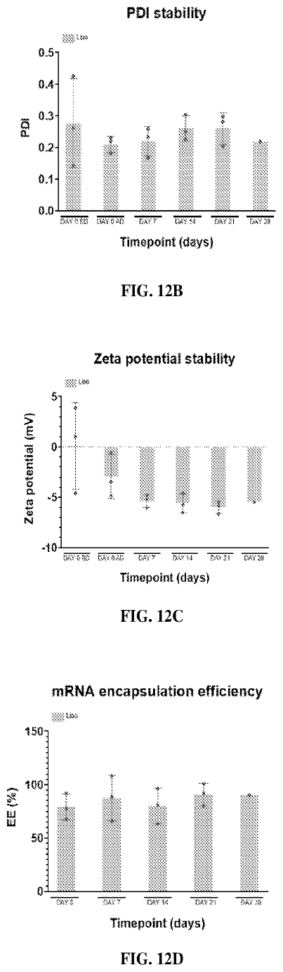

In vitro knockdown efficiency of 2 candidate siRNA-leukosome formulations: While the 2 siRNA-leukosome formulations of interest met the desired NP characteristics, validation of their knockdown efficiency was tested on 4T1 cells. While treatment with scramble siRNA-loaded NPs resulted in no change in STAT3 expression levels, both Lipo-STAT3 and Leuko-STAT3 did achieve knockdown to varying degrees. siRNA-NPs with mid PEG percentage exhibited up to a 75% knockdown in STAT3 mRNA expression within 48 h, with both liposomes and leukosomes outperforming the Lipofectamine control (FIG. 8A). In NPs synthesized with 6% of PEG, leukosomes demonstrated a 70% knockdown in STAT3 mRNA levels after 24 h, which was comparable to the Lipofectamine control (FIG. 8B). In contrast, liposomes with the same PEG content exhibited a significantly less knockdown efficiency, with only 35% of knockdown after 24 h (FIG. 8B).

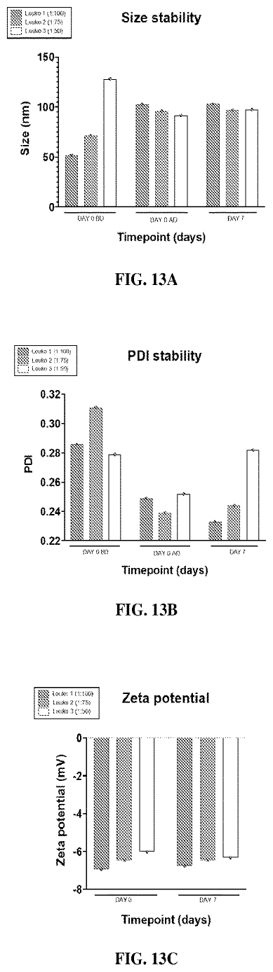

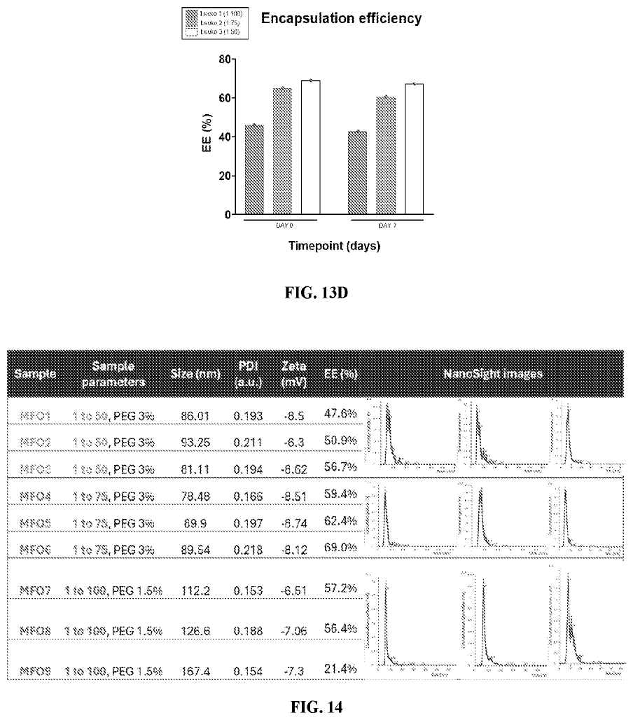

TNBC tumor targeting and biodistribution of 2 candidate siRNA-leukosome formulations: A 4T1 tumor model of TNBC was utilized for the evaluation of the biodistribution and tumor targeting properties of the selected siRNA-leukosome formulations. Whole organ ex vivo imaging following systemic administration of siRNA-liposomes and siRNA-leukosomes indicated maximum NP accumulation in the liver and tumor, with very minimal accumulation in other filtering organs for up to 6 h (FIGS. 9A-9B). When comparing the NP accumulation levels between the PEG mid and PEG high NPs, the latter exhibited higher levels of accumulation over the former, especially in the liver (FIGS. 9A-9B). At 1 h, liver accumulation of PEG High NPs was almost 2-3-fold greater than that observed in the PEG mid formulation (FIGS. 9A-9B). This observed trend continued at the 6 h timepoint, with an overall 50-60% reduction in the overall signal observed in the liver (FIGS. 9C-9D). Further analysis of the NP accumulation in the liver and tumor revealed interesting observations (FIGS. 10A-10D). NPs of both PEG ratios demonstrated similar levels of accumulation within the tumor, with little differences observed between the liposomes and leukosomes (FIG. 10A). The decrease in NP signal in the liver over time was indicative of NP clearance, while maintaining signal within the tumor (FIG. 10B). The latter observation required further analysis of the kinetics of NP behavior within the tumor environment.

### DISCUSSION

This study demonstrated the development of a leukosome formulation capable of being loaded with RNA molecules. Although the translation of the leukosome strategy was initially thought to be easily transferrable for this cargo payload, the initial results indicated that determination for the formulation required detailed analysis of the interplay between the individual components of the NPs in conjunction with the synthesis process itself.

The initial lipid screen performed for the identification of the lipid backbone proved vital for identifying the best candidate for the new siRNA-leukosome formulation. Although previous studies have established the use of these different lipids, the validation performed in this study further corroborated the superior properties of emerging lipids, like DLin-MC3-DMA, for genetic cargo delivery.

Selection of the lipid backbone was the first of many steps in the development of the siRNA-leukosome formulation. As the initial experiments suggested, incorporation of proteins and RNA into these lipid NPs perturbed the assembly of the particles from a typical lipid NP containing only RNA molecules. This was confirmed from both the physiochemical properties of the NPs and the visual difference between siRNA-liposomes and siRNA-leukosomes. The apparent size and PDI increase along with the opacity of the samples prior to the filtration process were the hard evidence corroborating the interfering role of the protein presence. From the physiochemical perspective, the size and PDI was successfully reduced following the filtration step. On the other hand, the loss of siRNA during this purification step indicated the loss of material, specifically NPs and the associated protein components on their surface hypothesized to mediate their targeting behavior in vivo. Nonetheless, this initial set of analysis proved vital for recognizing not only the need to analyze these NPs further, but also enabling an understanding of what key factors can be tuned to stabilize these particles during the synthesis process.

The tuning of the siRNA-NP formulation provided valuable insights on the effect of 2 key variables—PEG content and flow rate for mixing—on the final siRNA-leukosome formulation. In contrast to the previously known behavior of PEG in minimizing NP and cellular protein interactions, the results from this study indicated that the PEG presence was indeed necessary for the stabilization of the NPs in terms of size and PDI. This could be concluded from testing both extremes of PEG presence on the NPs—without any PEG lipid and 4-fold increase in PEG concentration. Furthermore, the decrease in loss of siRNA was indicative of improved packing of the NPs due to their smaller size and, thereby, facilitating their easy passage through the size cut-off of the utilized filter. This was further supported by the appearance of the solutions themselves where siRNA-leukosomes with highest PEG content were clear in appearance and closely resembling the siRNA-liposomes even prior to their filtration. This trend in the reduction of the size of siRNA-liposomes was also recently reported and aligns with the findings of this study[¹⁸].

In addition to the surprising results obtained while increasing the PEG content, tuning of the flow rate during synthesis also provided findings that were in contrast to the expected outcomes. Previous studies have corroborated the effect of increasing the flow rate when using a microfluidic-based synthesis method for the synthesis of liposomes, where a significant reduction in the size of the final NP was observed^{[19]}. However, the findings of this study suggest that this trend does not necessarily translate to these RNA-loaded lipid NPs. In fact, little effect was observed on the NP size when increasing the flow rate by almost 5-fold. Taken together, these results demonstrated how these variables of PEG content and flow ratio impact the self-assembly process of the NP formulation, especially in the incorporation of proteins and RNA in a lipid NP.

In vitro evaluation demonstrated the safety of the selected siRNA-leukosome formulations, with both formulations resulting in minimal cell death for up to 72 h. Knockdown efficiency profile of both siRNA-leukosome formulations performed comparable to the Lipofectamine control. Interestingly, this trend did not carry over to the equivalent siRNA-liposome formulations. The absence of knockdown following treatment with the siRNA-liposomes with mid PEG content was contradicted by that achieved when using same liposomes with higher PEG content. This specific finding warrants further study such as through analysis of the siRNA release profile from these individual formulations. From the in vivo perspective, both formulations of NPs demonstrated the maximum accumulation within the liver, which aligns with previous studies utilizing this lipid backbone for gene delivery^{[11]}. Of note was the difference observed in the levels of accumulation within the liver when increasing the PEG ratio, highlighting the underlying role of the PEG content in mediating the overall biodistribution of the NPs. From the perspective of tumor accumulation, the increased PEG content appears to support longer retention of the NPs within the tumor environment. On the other hand, the presence of the proteins in the siRNA-leukosomes did not demonstrate an improved targeting to the tumor site. The combined findings from the in vitro and in vivo analysis have emphasized the subsequent effects on the biological behavior of these NPs when tuning the PEG content.

The study has demonstrated the ability to synthesize siRNA lipid nanoparticles containing proteins. Most importantly, the findings of this study highlight the importance of deconvoluting the lipid self-assembly process and the variables that contribute to this process. This understanding was proven to be even more important when adding another component to this complex system, which are the membrane proteins of the leukosome formulation.

### Example 2: siRNA Payload

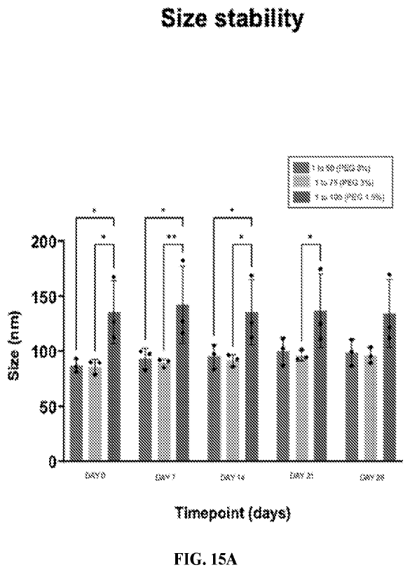

FIG. 11 shows a general schematic of the leukosomes. LNP formulations (n=3, no protein) were designed to improve siRNA encapsulation efficiency. The first iteration, which did not work, is shown in TABLE 2. The N/P ratio was 3, the flow rate ratio was 1:3, and the total flow rate was 5 mL/min.

TABLE 2
Iteration 1.

| Lipid | Molar % |
|---|---|
| DLin-MC3-DMA | 50% |
| Cholesterol | 33.5 |
| DSPC | 10.5 |
| DMG-PEG2000 | 6 |

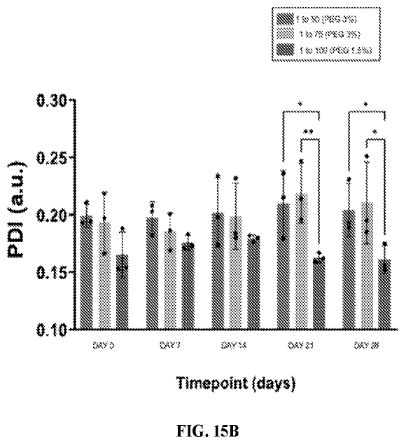

The results of iteration 1 is shown in FIGS. 11A-11D. LNP formulations (n=3) displayed higher encapsulation efficiency (>60%) compared to previous iterations (FIG. 12D), but the DMG-PEG2000 molar percentage of 6% led to a small size (~50 nm, FIG. 12A),which could cause quick clearance in vivo. Additional iterations can have a decreased DMG-PEG2000 molar %, even with the addition of membrane proteins. All the physio-chemical properties (Size, PDI—panel b, Zeta potential—FIG. 12C) were measured before dialysis (BD) and for up to 28 days post synthesis, upon storage at 4° C. Encapsulation efficiency was measured for up to 28 days in the same conditions. All parameters appear to be stable.

In the second iteration, LNP formulations (n=3, varying protein:lipid ratios) were designed to improve control over size. The change from the previous iteration was the molar percentage of DMG-PEG2000, decreased from 6% to 3% in all Leukosomal LNP formulations, independently of the lipid:protein ratio. The second iteration, which also did not work for the lipid:protein ratio of 1:100, is shown in TABLE 3. The N/P ratio was 3, the flow rate ratio was 1:3, and the total flow rate was 5 mL/min.

TABLE 3
Iteration 2.

| Protein:lipid 1:100 | | Protein:lipid 1:75 | | Protein:lipid 1:50 | |
|---|---|---|---|---|---|
| (Leuko 1) | | (Leuko 2) | | (Leuko 3) | |
| | Molar | | Molar | | Molar |
| Lipid | % | Lipid | % | Lipid | % |
| DLin-MC3- | 50 | DLin-MC3- | 50 | DLin-MC3- | 50 |
| DMA | | DMA | | DMA | |
| Cholesterol | 36.5 | Cholesterol | 36.5 | Cholesterol | 36.5 |
| DSPC | 10.5 | DSPC | 10.5 | DSPC | 10.5 |
| DMG-PEG2000 | 3 | DMG-PEG2000 | 3 | DMG-PEG2000 | 3 |

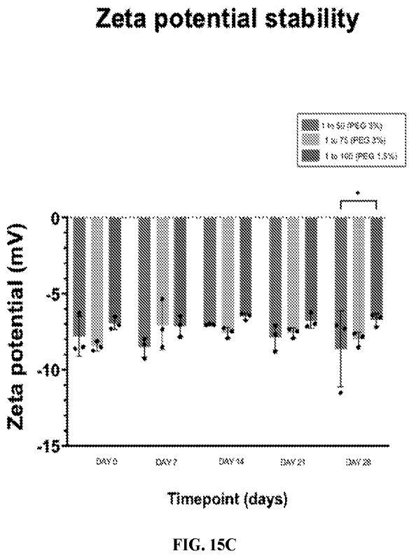

The results of iteration 2 are shown in FIGS. 12A-12D. Leukosome-LNP formulations had inconsistent size before dialysis (BD), but size values stabilized around −100 nm after dialysis (FIG. 13A). Because the size of Leukosome-LNPs with protein:lipid ratio of 1:100 (Leuko 1) was −50 nm, next iterations included a lower molar percentage of DMG-PEG2000.

In the third iteration, LNP formulations (varying protein:lipid ratios) were designed to improve control over size. The change from the previous iteration was the molar percentage of DMG-PEG2000, decreased from 3% to 1.5% in Leukosomal LNP formulations with lipid:protein ratio 1:100. To have control over the amount of Extraction buffer II introduced during synthesis, the J774 protein stock concentration was fixed at 4 mg/ml. The third iteration, which did work, is shown in TABLE 4. The N/P ratio was 3, the flow rate ratio was 1:3, the total flow rate was 5 mL/min, and the protein stock concentration was 4 mg/mL.

TABLE 4
Iteration 3.

| Protein:lipid 1:100 | | Protein:lipid 1:75 | | Protein:lipid 1:50 | |
|---|---|---|---|---|---|
| (MFO7, MFO8, MFO9) | | (MFO4, MFO5, MFO6) | | (MFO1, MFO2, MFO3) | |
| | Molar | | Molar | | Molar |
| Lipid | % | Lipid | % | Lipid | % |
| DLin-MC3- | 50 | DLin-MC3- | 50 | DLin-MC3- | 50 |
| DMA | | DMA | | DMA | |
| Cholesterol | 38 | Cholesterol | 36.5 | Cholesterol | 36.5 |
| DSPC | 10.5 | DSPC | 10.5 | DSPC | 10.5 |
| DMG-PEG2000 | 1.5 | DMG-PEG2000 | 3 | DMG-PEG2000 | 3 |

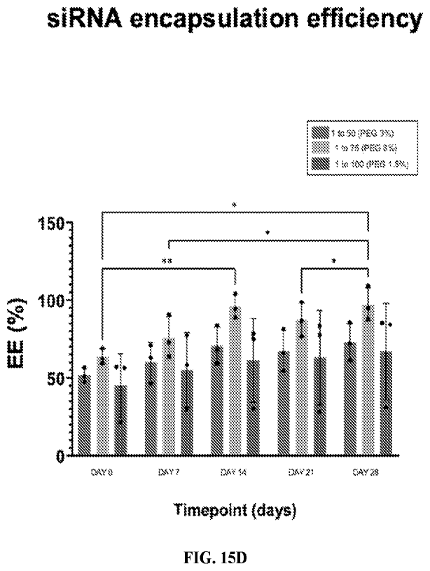

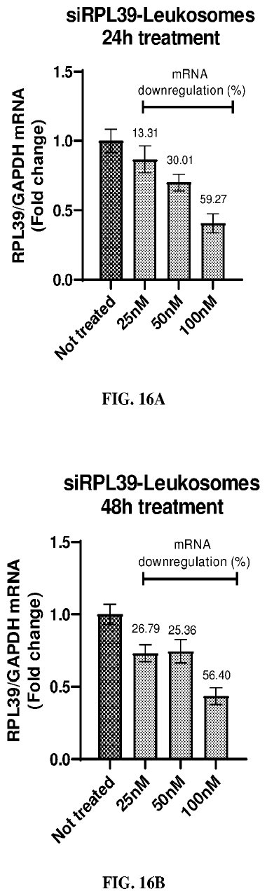

The results of iteration 3 are shown in FIG. 14 and FIGS. 14A-14D. FIG. 13 shows day 0 post synthesis. FIGS. 14A-14D show that the size (FIG. 15A) and PDI (FIG. 15B) of Leukosome-LNPs improved. The 1 to 75, PEG 3% LNPs seemed to be the most ideal in terms of size, PDI, Z-potential (FIG. 15C), and encapsulation efficiency (FIG. 15D). No relevant changes in parameters occurred over time, upon storage at 4° C.

To further investigate the Leukosome-LNP formulation with protein:lipid ratio of 1:75, two parameters were changed from iteration 3: the ionizable lipid was changed from Dlin-MC3-DMA to SM-102, and the total flow rate of the Benchtop Nanoassemblr was increased to 9 ml/min to improve the PDI. The finalized parameters, with and without the protein, are shown in TABLE 5 and TABLE 6. For both, the N/P ratio was 3, the flow rate ratio was 1:3, the total flow rate was 5 mL/min, and the ionizable lipid was SM-102. When the protein was incorporated, the protein stock concentration was 4 mg/mL.

TABLE 5
Iteration 4, no protein.
LNP (No protein)

| Lipid | Molar % |
|---|---|
| SM-102 | 50 |
| Cholesterol | 38 |
| DSPC | 10.5 |
| DMG-PEG2000 | 1.5 |

TABLE 6
Iteration 4.
Protein:lipid 1:75

| Lipid | Molar % |
|---|---|
| SM-102 | 50 |
| Cholesterol | 36.5 |
| DSPC | 10.5 |
| DMG-PEG2000 | 3 |

Characterization of the finalized nanovesicles is shown in TABLE 7. The implemented changes led to a decrease in PDI, suggesting a more uniform size distribution compared to previous iterations. Additionally, switching to the ionizable lipid SM-102 seems to have caused an improvement in encapsulation efficiency, now reaching 95.6% for regular LNPs, and above 89% for Leukosome-LNPs (protein:lipid ratio 1:75).

TABLE 7
Characterization of iteration 4.

| | Size | PDI | Zeta | Encapsulation |
|---|---|---|---|---|
| Formulation | (nm) | (a.u) | Potential (mV) | efficiency (%) |
| LNP (No protein) | 76.79 | 0.07 | −2.76 | 95.6 |
| Leukosome LNP 1:75 | 134.73 | 0.15 | −3.80 | 89.50 |

FIGS. 15A-15B show in vitro tests for siRPL39 Leukosomes-LNPs. Finalized Leukosome-LNP formulations were loaded with siRNA against RPL39 to evaluate downregulation in vitro. MDA-MB-231 cells were treated with different concentrations of siRNA (25 nM, 50 nM, and 100 nM), and RPL39 mRNA downregulation was measured at 24 hours and 48 hours, compared against an untreated control. RPL39 mRNA downregulation was proportional to treatment concentration, with the 100 nM siRNA concentration leading to over 50% downregulation at both timepoints (FIGS. 15A-15B).

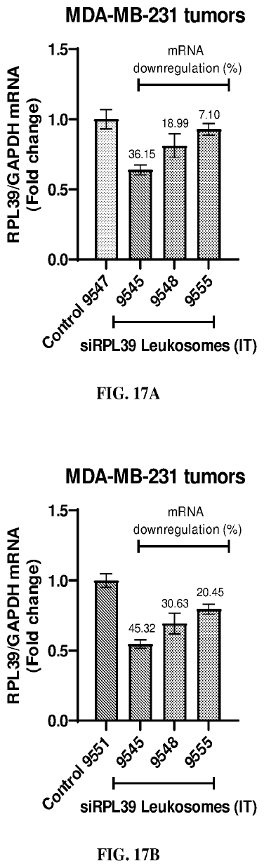

FIGS. 16A-16C show in vivo downregulation tests for siRPL39 Leukosomes-LNPs. 5×10⁶ MDA-MB-321 cells were injected in the mammary fat pad of NSG mice (xenografts). Once an average tumor size of 106.59 mm³ was reached, mice were randomized in the following treatment groups: (i) control (PBS, n=3, animal IDs: 9547, 9551, 9556), (ii) siRPL39 Leukosome-LNPs, intratumoral administration (IT, 1 μg/g) administration (n=3, animal IDs: 9545, 9548, 9555). 20 hours after treatment, animals were sacrificed to collect tumors for RNA extraction to measure RPL39 mRNA downregulation. Downregulation in siRPL39-treated tumors was compared to each control separately. Despite the variability in RPL39 mRNA expression within the animal model, it is possible to appreciate RPL39 mRNA downregulation in all siRPL30-treated animals (FIGS. 16A-16C).

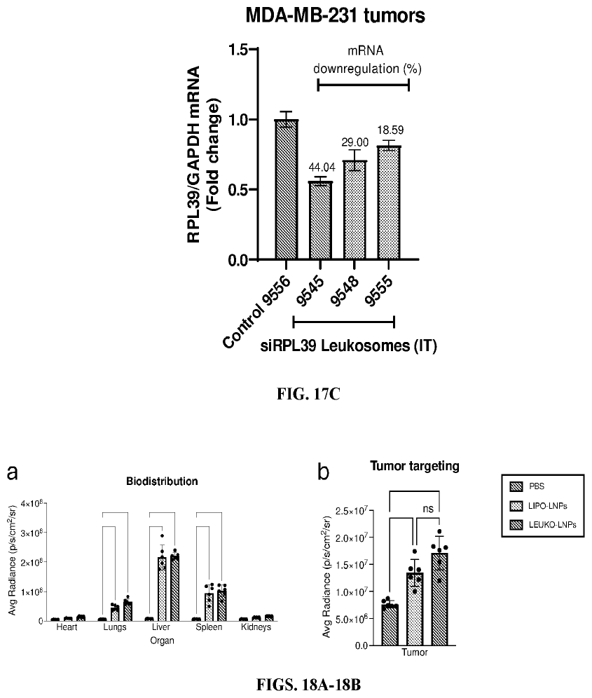

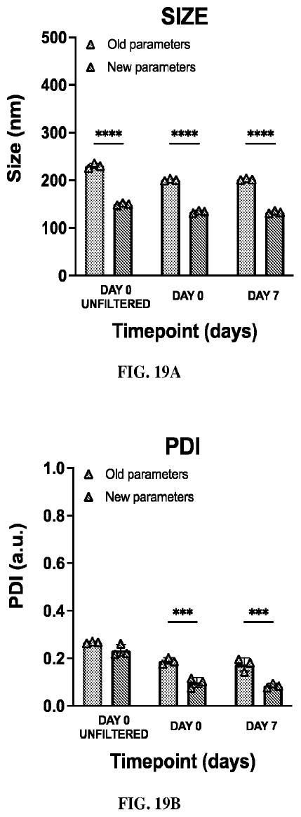

FIGS. 17A-17C show in vivo targeting tests for siRPL39 Leukosomes-LNPs. A metaplastic breast cancer model was developed with patient derived xenograft (PDX4664, RPL39 A14V). Once an average tumor size of ~100 mm³ was reached, animals were randomized in the three following treatment groups: (i) PBS (n=6), (ii) siRPL39 LNPs (n=6, 25 μg/mouse), (iii) siRPL39 Leukosome-LNPs (n=6, 25 μg/mouse). All treatments were administered intravenously (IV) and, 6 hours post treatment, animals were sacrificed to collect tumors and organs for a biodistribution assessment with IVIS. The biodistribution profile for IV administered LNPs showed predominantly hepatic accumulation (FIG. 18A). Despite the absence of significance, it is possible to appreciate an increased trend of Leukosome-LNPs for tumor accumulation, when compared to bare LNPs (FIG. 18B).

### Example 3: mRNA Payload

The leukosomes depicted in FIG. 11 were next developed to carry an mRNA payload. Leukosome-LNP formulations were designed to improve quality attributes of the final product (physio-chemical properties and encapsulation efficiency). These experiments are comparing two different Leukosome-LNP formulations (protein:lipid ratio 1:100): (i) “Old parameters” (shown in TABLE 8) refer to the formulation and synthesis parameters that are used to synthesize regular LNPs (no membrane proteins) when tested with the protein:lipid ratio of 1:100, and (ii) “New parameters” (shown in TABLE 9) refer to the implemented improvements. For the old parameters, the N/P ratio was 5.6, the flow rate ratio was 1:3, the total flow rate was 2 mL/min, and the protein source was membrane proteins from J774 murine macrophages. For the new parameters, the N/P ratio was 5.6, the flow rate ratio was 1:3, the total flow rate was 5 mL/min, and the protein source was membrane proteins from J774 murine macrophages.

TABLE 8
Old parameters.
Old parameters, Leukosome-LNPs (protein:lipid ratio 1:100)

| Lipid | Molar % |
|---|---|
| DLin-MC3-DMA | 50 |
| Cholesterol | 38 |
| DSPC | 10.5 |
| DMG-PEG2000 | 1.5 |

TABLE 9
New parameters.
New parameters, Leukosome-LNPs (protein:lipid ratio 1:100)

| Lipid | Molar % |
|---|---|
| DLin-MC3-DMA | 50 |
| Cholesterol | 36.5 |
| DSPC | 10.5 |
| DMG-PEG2000 | 3 |

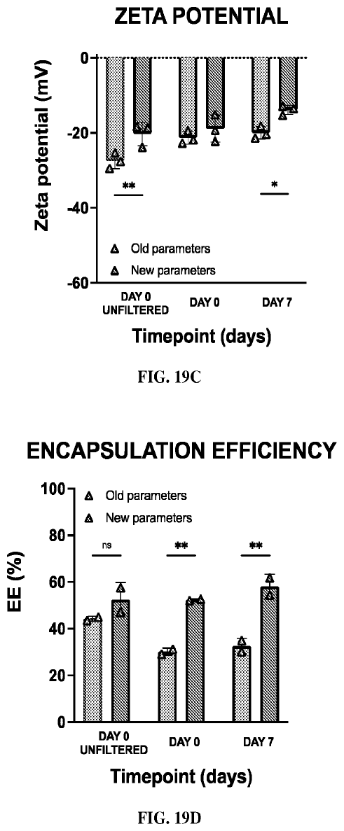

The results of iteration 1 are shown in FIGS. 18A-18D. Physio-chemical properties (size—FIG. 19A, PDI—FIG. 19B, Zeta potential—FIG. 19C) and encapsulation efficiency (EE—FIG. 19D) were followed with DLS & Ribogreen for 7 days after synthesis of LNPs loaded with Luciferase mRNA. After filtration with 0.22 μm filters, the “New parameters” (higher flow rates and increased DMG-PEG2000 molar percentages) improved physio-chemical properties and EE.

A surgically induced Post Traumatic Osteoarthritis (PTOA) murine model known ad Disruption of Medial Meniscus (DMM) was implemented to test the targeting potential of intravenously administered Leukosome-LNPs towards joint inflammation in the acute phase post injury, as well as their biodistribution. C57BL/j mice received a surgically induced injury on the right knee joint and, 24 hours post surgery, they were intravenously (IV) administered DiD-labeled Leukosome-LNPs loaded with Luciferase mRNA. 6 hours post-administration, animals were sacrificed to assess Leukosome-LNP biodistribution and targeting to the injured leg, as well as successful translation of the cargo (Luciferase is bioluminescent) with IVIS. Each mouse (n=5) received 40 μg of Luciferase mRNA.

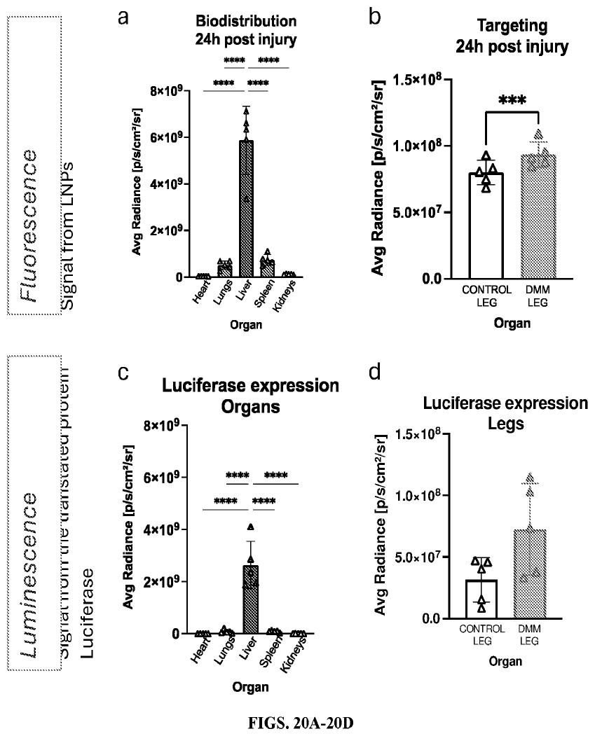

The in vivo biodistribution of Leukosome-LNPs is shown in FIGS. 19A-19D. Biodistribution profile of Leukosome-LNPs was predominantly hepatic (FIG. 20A), with some accumulation in spleen and lungs. Significantly higher accumulation of Leukosome-LNPs occurred in the injured DMM leg when compared to the control leg (FIG. 20B), confirming the targeting potential of Leukosome-LNPs in a PTOA murine model. Additionally, Leukosome-LNPs allowed successful translation of mRNA in vivo, the Luciferase signal having a similar profile to the one of LNP biodistribution (FIGS. 19C-19D) as shown by the luminescence signal.

In the second iteration, Leukosome-LNP formulations were designed to improve quality attributes of the final product (physio-chemical properties and encapsulation efficiency). From the previous iteration, changes involved the use of a different ionizable lipid (SM-102 instead of Dlin-MC3-DMA), an increase in N/P ratio (from 5.6 to 6), a change in molar percentage of DMG-PEG2000 (from 3% to 3.5%), and an increase in the total flow rate used for synthesis (from 5 ml/min to 12 ml/min). The implemented changes successfully led to a decrease in size (closer to ~100 nm) and PDI, leading to a more homogeneous size distribution. Additionally, encapsulation efficiency (EE %) was improved, reaching 80%. The finalized parameters are shown in TABLE 10, and the characterization is shown in TABLE 11. The ionizable lipid was SM-102, the N/P ratio was 6, the flow rate ratio was 1:3, the total flow rate was 12 mL/min, and the protein source was membrane proteins from J774 murine macrophages.

TABLE 10
Iteration 2.
Leukosome-LNPs (protein:lipid ratio 1:100)

| Lipid | Molar % |
|---|---|
| SM-102 | 50 |
| Cholesterol | 36 |
| DSPC | 10.5 |
| DMG-PEG2000 | 3.5 |

TABLE 11
Characterization of iteration 2.

| Size | PDI | ZP | RNA Encapsulation |
|---|---|---|---|
| (nm) | (a.u.) | (mV) | efficiency (EE %) |
| 113.67 | 0.17 | −13.9 | 80 |

Leukosome-LNPs synthesized with the parameters defined in Iteration 2 were used to evaluate in vivo biodistribution and targeting in a Triple Negative Breast Cancer (TNBC) murine model. Female BalbC mice (2 months old) received a mammary fat pad injection of 3×10⁵ 4T1 cells and, once tumors reached an apparent size of 50-80 mm³, they were randomized in the following groups: (i) intravenous (IV) injection of DiD-Labeled LNPs (No membrane proteins, n=6), (ii) IV injection of DiD-Labeled Leukosome-LNPs (protein:lipid 1:100, n=6), (iii) subcutaneous (SC) injection of DiD-Labeled LNPs (No membrane proteins, n=6), and (iv) SC injection of DiD-Labeled Leukosome-LNPs (protein:lipid 1:100, n=6). 6 hours post administration, all animals were sacrificed to collect organs and tumors to measure LNP accumulation via IVIS.

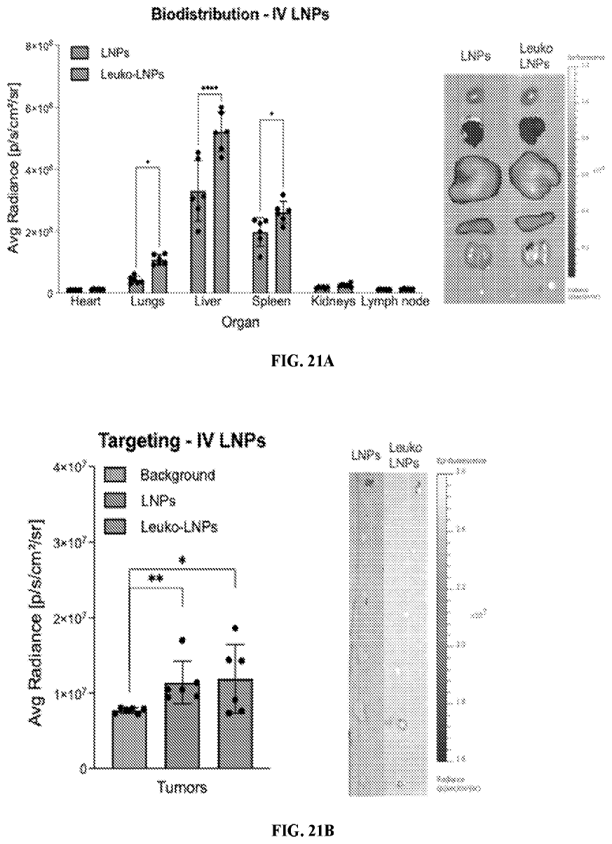

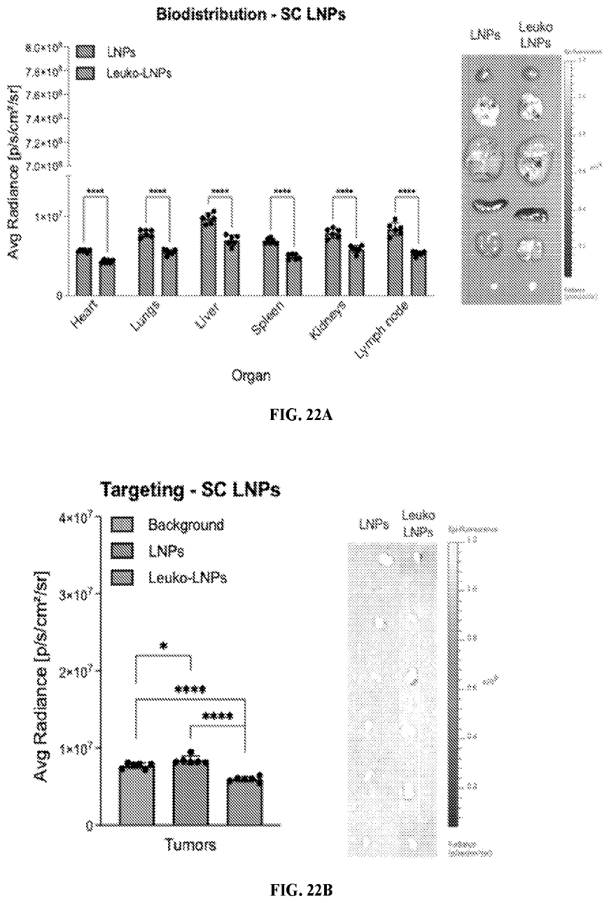

The results of in vivo biodistribution and targeting of iteration 2 is shown in FIGS. 20A-20B and FIGS. 21A-21B. IV administered LNPs had predominantly hepatic accumulation (FIG. 21A), consistent with previous results. A trend of increased tumor accumulation was identified for Leukosome-LNPs when compared with regular LNPs (FIG. 21B), despite the absence of significance. SC administered LNPs did not move from the injection area, as demonstrated by the absence of signal in organs (FIG. 22A) and tumors (FIG. 22B). This suggests that targeting cannot be achieved via SC administration of Leukosome-LNPs.

In iteration 3, the Leukosome-LNP formulation was tested to evaluate feasibility of using a different lipidic composition while maintaining acceptable “quality attributes” of the formulation. From the previous iteration (Iteration 2), changes involved the following items: (i) the use of a different ionizable lipid (ALC-0315 instead of SM-102) and a difference in its molar %, (ii) the use of two different helper lipids (DOPG and PS instead of DSPC), (iii) the use of a different pegylated lipid (ALC-0159 instead of DMG-PEG2000) and an increase in its molar % (from 3.5% to 4%). Changes in the helper lipid composition were implemented to improve translation efficiency of the mRNA. The finalized parameters are shown in TABLE 12, and the characterization is shown in TABLE 13. While quality attributes were maintained for what pertains size, PDI, and Zeta Potential (ZP), the encapsulation efficiency slightly decreased. This may be due to the fact that this iteration involved the use of a synthetic recombinant protein instead of membrane proteins extracted from J774 murine macrophages. The ionizable lipid was ALC-0315, the helper lipids were DOPG (1,2-dioleoyl-sn-glycero-3-phospho-(1′-rac-glycerol) (sodium salt)) and Brain PS (L-α-phosphatidylserine (Brain, Porcine) (sodium salt)), the PEGylated lipid was ALC-0159, the N/P ratio was 6, the flow rate ratio was 1:3, the total flow rate was 12 mL/min, the protein:lipid ratio was 1:90, and the protein source was recombinant protein LFA1.

TABLE 12
Iteration 3.

| Lipid | Molar % |
|---|---|
| ALC-0315 | 46.3 |
| Cholesterol | 40.3 |
| DOPG | 6.9 |
| Brain PS | 2.5 |
| ALC-0159 | 4 |

TABLE 13
Characterization of iteration 3.

| Size | PDI | ZP | Encapsulation |
|---|---|---|---|
| (nm) | (a.u.) | (mV) | efficiency (EE %) |
| 82.31 | 0.16 | −18.3 | 40 |

The following patents, applications and publications as listed below and throughout this document are hereby incorporated by reference in their entirety herein.

### REFERENCE LIST

Alexis, F.; Pridgen, E.; Molnar, L. K.; Farokhzad, 0. C., Factors Affecting the Clearance and Biodistribution of Polymeric Nanoparticles. Mol Pharm 2008, 5, 505-515.
Alkilany, A. M.; Zhu, L.; Weller, H.; Mews, A.; Parak, W. J.; Barz, M.; Feliu, N., Ligand Density on Nanoparticles: A Parameter with Critical Impact on Nanomedicine. Adv Drug Deliv Rev 2019, 143, 22-36.
Andrew Brown, A. T., Mina Ordobadi, Shell Ip, Gesine Heuck, Euan Ramsay, Liposomes: Using the NanoAssemblr Benchtop instrument process parameters to reproducibly tune size. 2018, Precision NanoSystems.
Arango Duque, G.; Descoteaux, A., Macrophages Tell the Non-Professionals What to Do. Dev Cell 2016, 39, 633-635.
Ben-Akiva, E.; Meyer, R. A.; Yu, H.; Smith, J. T.; Pardoll, D. M.; Green, J. J., Biomimetic Anisotropic Polymeric Nanoparticles Coated with Red Blood Cell Membranes for Enhanced Circulation and Toxin Removal. Science Advances 2020, 6, eaay9035.
Blanco, E.; Shen, H.; Ferrari, M., Principles of Nanoparticle Design for Overcoming Biological Barriers to Drug Delivery. Nat Biotechnol 2015, 33, 941-951.
Bunger, S.; Roblick, U. J.; Habermann, J. K., Comparison of Five Commercial Extraction Kits for Subsequent Membrane Protein Profiling. Cytotechnology 2009, 61, 153-159.
Cheng, Q., et al., Selective organ targeting (SORT) nanoparticles for tissue-specific mRNA delivery and CRISPR-Cas gene editing. Nature Nanotechnology, 2020. 15(4): p. 313-320.
Cullis, P. R. and M. J. Hope, Lipid Nanoparticle Systems for Enabling Gene Therapies. Molecular Therapy, 2017. 25(7): p. 1467-1475.
Danaei, M.; Dehghankhold, M.; Ataei, S.; Hasanzadeh Davarani, F.; Javanmard, R.; Dokhani, A.; Khorasani, S.; Mozafari, M. R., Impact of Particle Size and Polydispersity Index on the Clinical Applications of Lipidic Nanocarrier Systems. Pharmaceutics 2018, 10, 57.
de Fougerolles, A. R., Delivery vehicles for small interfering RNA in vivo. Hum Gene Ther, 2008. 19(2): p. 125-32.
Dimov, N.; Kastner, E.; Hussain, M.; Perrie, Y.; Szita, N., Formation and Purification of Tailored Liposomes for Drug Delivery Using a Module-Based Micro Continuous-Flow System. Sci Rep 2017, 7, 12045.
   Dudley, A. C., Tumor Endothelial Cells. Cold Spring Harb Perspect Med 2012, 2, a006536.
Gimbrone, M. A., Jr.; Garcia-Cardena, G., Endothelial Cell Dysfunction and the Pathobiology of Atherosclerosis. Circ Res 2016, 118, 620-636.
Hattab, D. and A. Bakhtiar, Bioengineered siRNA-Based Nanoplatforms Targeting Molecular Signaling Pathways for the Treatment of Triple Negative Breast Cancer: Preclinical and Clinical Advancements. Pharmaceutics, 2020. 12(10).
Hu, C. M.; Fang, R. H.; Copp, J.; Luk, B. T.; Zhang, L., A Biomimetic Nanosponge That Absorbs Pore-Forming Toxins. Nat Nanotechnol 2013, 8, 336-340.
Huynh, A., C. O. Madu, and Y. Lu, siRNA: A Promising New Tool for Future Breast Cancer Therapy. Oncomedicine, 2018. 3: p. 74-81.
Jayaraman, M., et al., Maximizing the Potency of siRNA Lipid Nanoparticles for Hepatic Gene Silencing In vivo. Angewandte Chemie International Edition, 2012. 51(34): p. 8529-8533.
Jiang, Y., et al., Progress and perspective of inorganic nanoparticle-based siRNA delivery systems. Expert Opin Drug Deliv, 2016. 13(4): p. 547-59.
Kaczmarek, J. C., P. S. Kowalski, and D. G. Anderson, Advances in the delivery of RNA therapeutics: from concept to clinical reality. Genome Med, 2017. 9(1): p. 60.
Khalil, I. A., et al., Lipid Nanoparticles for Cell-Specific in vivo Targeted Delivery of Nucleic Acids. Biol Pharm Bull, 2020. 43(4): p. 584-595.
Kim, M., et al., Engineered ionizable lipid nanoparticles for targeted delivery of RNA therapeutics into different types of cells in the liver. Science Advances, 2021. 7(9): p. eabf4398.
Kozielski, K. L., S. Y. Tzeng, and J. J. Green, Bioengineered nanoparticles for siRNA delivery. Wiley Interdiscip Rev Nanomed Nanobiotechnol, 2013. 5(5): p. 449-68.
Ley, K., Molecular Mechanisms of Leukocyte Recruitment in the Inflammatory Process. Cardiovasc Res 1996, 32, 733-742.
Ley, K.; Laudanna, C.; Cybulsky, M. I.; Nourshargh, S., Getting to the Site of Inflammation: The Leukocyte Adhesion Cascade Updated. Nat Rev Immunol 2007, 7, 678-689.
Lorenzer, C., et al., Going beyond the liver: Progress and challenges of targeted delivery of siRNA therapeutics. Journal of Controlled Release, 2015. 203: p. 1-15.
Martinez, J. O.; Molinaro, R.; Hartman, K. A.; Boada, C.; Sukhovershin, R.; De Rosa, E.; Kirui, D.; Zhang, S.; Evangelopoulos, M.; Carter, A. M.; Bibb, J. A.; Cooke, J. P.; Tasciotti, E., Biomimetic Nanoparticles with Enhanced Affinity Towards Activated Endothelium as Versatile Tools for Theranostic Drug Delivery. Theranostics 2018, 8, 1131-1145.
Molinaro, R.; Corbo, C.; Martinez, J. O.; Taraballi, F.; Evangelopoulos, M.; Minardi, S.; Yazdi, I. K.; Zhao, P.; De Rosa, E.; Sherman, M. B.; De Vita, A.; Toledano Furman, N. E.; Wang, X.; Parodi, A.; Tasciotti, E., Biomimetic Proteolipid Vesicles for Targeting Inflamed Tissues. Nat Mater 2016, 15, 1037-1046.
Molinaro, R.; Evangelopoulos, M.; Hoffman, J. R.; Corbo, C.; Taraballi, F.; Martinez, J. O.; Hartman, K. A.; Cosco, D.; Costa, G.; Romeo, I.; Sherman, M.; Paolino, D.; Alcaro, S.; Tasciotti, E., Design and Development of Biomimetic Nanovesicles Using a Microfluidic Approach. Adv Mater 2018, 30, e1702749.
Molinaro, R.; Martinez, J. O.; Zinger, A.; De Vita, A.; Storci, G.; Arrighetti, N.; De Rosa, E.; Hartman, K. A.; Basu, N.; Taghipour, N.; Corbo, C.; Tasciotti, E., Leukocyte-Mimicking Nanovesicles for Effective Doxorubicin Delivery to Treat Breast Cancer and Melanoma. Biomater Sci 2020, 8, 333-341.
Nourshargh, S.; Alon, R., Leukocyte Migration into Inflamed Tissues. Immunity 2014, 41, 694-707.
Parodi, A.; Molinaro, R.; Sushnitha, M.; Evangelopoulos, M.; Martinez, J. O.; Arrighetti, N.; Corbo, C.; Tasciotti, E., Bio-Inspired Engineering of Cell- and Virus-Like Nanoparticles for Drug Delivery. Biomaterials 2017, 147, 155-168.
Patra, J. K.; Das, G.; Fraceto, L. F.; Campos, E. V. R.; Rodriguez-Torres, M. d. P.; Acosta-Torres, L. S.; Diaz-Torres, L. A.; Grillo, R.; Swamy, M. K.; Sharma, S.; Habtemariam, S.; Shin, H.-S., Nano Based Drug Delivery Systems: Recent Developments and Future Prospects. Journal of Nanobiotechnology 2018, 16, 71.
Pelaz, B.; del Pino, P.; Maffre, P.; Hartmann, R.; Gallego, M.; Rivera-Fernandez, S.; de la Fuente, J. M.; Nienhaus, G. U.; Parak, W. J., Surface Functionalization of Nanoparticles with Polyethylene Glycol: Effects on Protein Adsorption and Cellular Uptake. ACS Nano 2015, 9, 6996-7008.
Roberts, T. C., R. Langer, and M. J. A. Wood, Advances in oligonucleotide drug delivery. Nature Reviews Drug Discovery, 2020. 19(10): p. 673-694.
Rodriguez, P. L.; Harada, T.; Christian, D. A.; Pantano, D. A.; Tsai, R. K.; Discher, D. E., Minimal” Self” Peptides That Inhibit Phagocytic Clearance and Enhance Delivery of Nanoparticles. Science 2013, 339, 971-975.
Rodriguez, P. L.; Harada, T.; Christian, D. A.; Pantano, D. A.; Tsai, R. K.; Discher, D. E., Minimal “Self” Peptides That Inhibit Phagocytic Clearance and Enhance Delivery of Nanoparticles. Science 2013, 339, 971-975.
Ryals, R. C., et al., The effects of PEGylation on LNP based mRNA delivery to the eye. PLoS One, 2020. 15(10): p. e0241006.
Schouten, M.; Wiersinga, W. J.; Levi, M.; van der Poll, T., Inflammation, Endothelium, and Coagulation in Sepsis. J Leukoc Biol 2008, 83, 536-545.
Sroda, K.; Rydlewski, J.; Langner, M.; Kozubek, A.; Grzybek, M.; Sikorski, A. F., Repeated Injections of Peg-Pe Liposomes Generate Anti-Peg Antibodies. Cell Mol Biol Lett 2005, 10, 37-47.
Stanimirovic, D.; Satoh, K., Inflammatory Mediators of Cerebral Endothelium: A Role in Ischemic Brain Inflammation. Brain Pathol 2000, 10, 113-126.
Sun, H.; Su, J.; Meng, Q.; Yin, Q.; Chen, L.; Gu, W.; Zhang, P.; Zhang, Z.; Yu, H.; Wang, S.; Li, Y., Cancer-Cell-Biomimetic Nanoparticles for Targeted Therapy of Homotypic Tumors. Adv Mater 2016, 28, 9581-9588.
Sushnitha, M.; Evangelopoulos, M.; Tasciotti, E.; Taraballi, F., Cell Membrane-Based Biomimetic Nanoparticles and the Immune System: Immunomodulatory Interactions to Therapeutic Applications. Frontiers in Bioengineering and Biotechnology 2020, 8, 627.
Vestweber, D., How Leukocytes Cross the Vascular Endothelium. Nat Rev Immunol 2015, 15, 692-704.
Walsh, C., et al., Microfluidic-based manufacture of siRNA-lipid nanoparticles for therapeutic applications. Methods Mol Biol, 2014. 1141: p. 109-20.
Witzigmann, D., et al., Lipid nanoparticle technology for therapeutic gene regulation in the liver. Adv Drug Deliv Rev, 2020. 159: p. 344-363.
Wu, J., et al., Binding characteristics between polyethylene glycol (PEG) and proteins in aqueous solution. Journal of Materials Chemistry B, 2014. 2(20): p. 2983-2992.
Xie, W.; Deng, W. W.; Zan, M.; Rao, L.; Yu, G. T.; Zhu, D. M.; Wu, W. T.; Chen, B.; Ji, L. W.; Chen, L.; Liu, K.; Guo, S. S.; Huang, H. M.; Zhang, W. F.; Zhao, X.; Yuan, Y.; Dong, W.; Sun, Z. J.; Liu, W., Cancer Cell Membrane Camouflaged Nanoparticles to Realize Starvation Therapy Together with Checkpoint Blockades for Enhancing Cancer Therapy. ACS Nano 2019, 13, 2849-2857.
Zinger, A.; Baudo, G.; Naoi, T.; Giordano, F.; Lenna, S.; Massaro, M.; Ewing, A.; Kim, H. R.; Tasciotti, E.; Yustein, J. T.; Taraballi, F., Reproducible and Characterized Method for Ponatinib Encapsulation into Biomimetic Lipid Nanoparticles as a Platform for Multi-Tyrosine Kinase-Targeted Therapy. ACS Applied Bio Materials 2020, 3, 6737-6745.
Zinger, A.; Brozovich, A.; Pasto, A.; Sushnitha, M.; Martinez, J. O.; Evangelopoulos, M.; Boada, C.; Tasciotti, E.; Taraballi, F., Bioinspired Extracellular Vesicles: Lessons Learned from Nature for Biomedicine and Bioengineering. Nanomaterials (Basel) 2020, 10, 2172.
Zinger, A.; Koren, L.; Adir, O.; Poley, M.; Alyan, M.; Yaari, Z.; Noor, N.; Krinsky, N.; Simon, A.; Gibori, H.; Krayem, M.; Mumblat, Y.; Kasten, S.; Ofir, S.; Fridman, E.; Milman, N.; Lubtow, M. M.; Liba, L.; Shklover, J.; Shainsky-Roitman, J. et al., Collagenase Nanoparticles Enhance the Penetration of Drugs into Pancreatic Tumors. ACS Nano 2019, 13, 11008-11021.
Zook, J. M.; Vreeland, W. N., Effects of Temperature, Acyl Chain Length, and Flow-Rate Ratio on Liposome Formation and Size in a Microfluidic Hydrodynamic Focusing Device. Soft Matter 2010, 6, 1352-1360.
Zook, J. M. and W. N. Vreeland, Effects of temperature, acyl chain length, and flow-rate ratio on liposome formation and size in a microfluidic hydrodynamic focusing device. Soft Matter, 2010. 6(6): p. 1352-1360.

What is claimed is:

1. A biomimetic proteolipid nanovesicle, comprising:
an ionizable or cationic lipid;
a phosphocholine-based phospholipid;
a cholesterol;
a leukocyte membrane protein; and
an siRNA encapsulated by the biomimetic proteolipid nanovesicle;
wherein the biomimetic proteolipid nanovesicle has a lipid-to-protein ratio of from about 1:65 to about 1:85 by weight.

2. The biomimetic proteolipid nanovesicle of claim 1, wherein the ionizable or cationic lipid is selected from the group consisting of DLin-MC3-DMA, SM-102, ALC-0315, and any combination thereof.

3. The biomimetic proteolipid nanovesicle of any one of claims 1-2, further comprising from about 40 molar % to about 60 molar % of the ionizable or cationic lipid.

4. The biomimetic proteolipid nanovesicle of claim 3, further comprising about 50 molar % of the ionizable or cationic lipid.

5. The biomimetic proteolipid nanovesicle of any one of claims 1-4, wherein the phosphocholine-based phospholipid is selected from the group consisting of phosphatidylcholine, egg phosphatidic acid, 1,2-dioleoyl-sn-glycerophosphocholine (DOPC), 1,2-diolyl-sn-lycerophosphoethanolamine (DOPE), 1,2-dipalmitoyl-sn-glycerophosphocholine (DPPC), 1,2-distearoyl-sn-glycerophosphocholine (DSPC), L-α-phosphatidylserine (PS), 1,2-dioleoyl-sn-glycero-3-phospho-(1′-rac-glycerol) (DOPG), and any combination thereof.

6. The biomimetic proteolipid nanovesicle of any one of claims 1-5, further comprising from about 7 molar % to about 15 molar % of the phosphocholine-based phospholipid.

7. The biomimetic proteolipid nanovesicle of claim 6, further comprising about 10.5 molar % of the phosphocholine-based phospholipid.

8. The biomimetic proteolipid nanovesicle of any one of claims 1-7, further comprising from about 30 molar % to about 40 molar % of the cholesterol.

9. The biomimetic proteolipid nanovesicle of claim 8, further comprising about 36.5 molar % of the cholesterol.

10. The biomimetic proteolipid nanovesicle of any one of claims 1-9, further comprising a PEGylated lipid.

11. The biomimetic proteolipid nanovesicle of claim 10, wherein the PEGylated lipid is selected from the group consisting of DMG-PEG2000, ALC-0159, and any combination thereof.

12. The biomimetic proteolipid nanovesicle of any one of claims 10-11, further comprising from about 1 molar % to about 10 molar % of the PEGylated lipid.

13. The biomimetic proteolipid nanovesicle of claim 12, further comprising about 3 molar % of the PEGylated lipid.

14. The biomimetic proteolipid nanovesicle of any one of claims 1-13, wherein the leukocyte membrane protein is derived from a leukocyte plasma membrane.

15. The biomimetic proteolipid nanovesicle of claim 14, wherein the leukocyte plasma membrane is a human leukocyte plasma membrane.

16. The biomimetic proteolipid nanovesicle of any one of claims 1-15, wherein the leukocyte membrane protein is a synthetic recombinant protein.

17. The biomimetic proteolipid nanovesicle of any one of claims 1-16, further comprising an N/P ratio of from about 1 to about 6.

18. The biomimetic proteolipid nanovesicle of claim 17, further comprising an N/P ratio of about 3.

19. The biomimetic proteolipid nanovesicle of any one of claims 1-18, wherein the biomimetic proteolipid nanovesicle has a diameter of from about 50 nm to about 200 nm.

20. The biomimetic proteolipid nanovesicle of claim 19, wherein the biomimetic proteolipid nanovesicle has a diameter of about 100 nm.

21. The biomimetic proteolipid nanovesicle of any one of claims 1-20, wherein the biomimetic proteolipid nanovesicle has a lipid-to-protein ratio of about 1:75 by weight.

22. The biomimetic proteolipid nanovesicle of any one of claims 1-21, wherein the siRNA is targeted to treat a cancer, inflammation an infectious disease, or a genetic disease or disorder.

23. A biomimetic proteolipid nanovesicle, comprising:
an ionizable or cationic lipid;
a phosphocholine-based phospholipid;
a cholesterol;
a leukocyte membrane protein; and
an mRNA encapsulated by the biomimetic proteolipid nanovesicle;
wherein the biomimetic proteolipid nanovesicle has a lipid-to-protein ratio of from about 1:80 to about 1:100 by weight.

24. The biomimetic proteolipid nanovesicle of claim 23, wherein the ionizable or cationic lipid is selected from the group consisting of DLin-MC3-DMA, SM-102, ALC-0315, and any combination thereof.

25. The biomimetic proteolipid nanovesicle of any one of claims 23-24, further comprising from about 40 molar % to about 60 molar % of the ionizable or cationic lipid.

26. The biomimetic proteolipid nanovesicle of claim 25, further comprising about 46 molar % of the ionizable or cationic lipid.

27. The biomimetic proteolipid nanovesicle of any one of claims 23-26, wherein the phosphocholine-based phospholipid is selected from the group consisting of phosphatidylcholine, egg phosphatidic acid, 1,2-dioleoyl-sn-glycerophosphocholine (DOPC), 1,2-diolyl-sn-lycerophosphoethanolamine (DOPE), 1,2-dipalmitoyl-sn-glycerophosphocholine (DPPC), 1,2-distearoyl-sn-glycerophosphocholine (DSPC), L-α-phosphatidylserine (PS), 1,2-dioleoyl-sn-glycero-3-phospho-(1′-rac-glycerol) (DOPG), and any combination thereof.

28. The biomimetic proteolipid nanovesicle of any one of claims 23-27, further comprising from about 7 molar % to about 15 molar % of the phosphocholine-based phospholipid.

29. The biomimetic proteolipid nanovesicle of claim 28, further comprising about 9.5 molar % of the phosphocholine-based phospholipid.

30. The biomimetic proteolipid nanovesicle of any one of claims 23-29, further comprising from about 35 molar % to about 45 molar % of the cholesterol.

31. The biomimetic proteolipid nanovesicle of claim 30, further comprising about 40.5 molar % of the cholesterol.

32. The biomimetic proteolipid nanovesicle of any one of claims 23-31, further comprising a PEGylated lipid.

33. The biomimetic proteolipid nanovesicle of claim 32, wherein the PEGylated lipid is selected from the group consisting of DMG-PEG2000, ALC-0159, and any combination thereof.

34. The biomimetic proteolipid nanovesicle of any one of claims 32-33, further comprising from about 1 molar % to about 10 molar % of the PEGylated lipid.

35. The biomimetic proteolipid nanovesicle of claim 34, further comprising about 4 molar % of the PEGylated lipid.

36. The biomimetic proteolipid nanovesicle of any one of claims 23-35, wherein the leukocyte membrane protein is derived from a leukocyte plasma membrane.

37. The biomimetic proteolipid nanovesicle of claim 36, wherein the leukocyte plasma membrane is a human leukocyte plasma membrane.

38. The biomimetic proteolipid nanovesicle of any one of claims 23-37, wherein the leukocyte membrane protein is a synthetic recombinant protein.

39. The biomimetic proteolipid nanovesicle of any one of claims 23-38, further comprising an N/P ratio of from about 3 to about 10.

40. The biomimetic proteolipid nanovesicle of claim 39, further comprising an N/P ratio of about 6.

41. The biomimetic proteolipid nanovesicle of any one of claims 23-40, wherein the biomimetic proteolipid nanovesicle has a diameter of from about 50 nm to about 200 nm.

42. The biomimetic proteolipid nanovesicle of claim 41, wherein the biomimetic proteolipid nanovesicle has a diameter of about 100 nm.

43. The biomimetic proteolipid nanovesicle of any one of claims 23-42, wherein the biomimetic proteolipid nanovesicle has a lipid-to-protein ratio of about 1:90 by weight.

44. The biomimetic proteolipid nanovesicle of any one of claims 23-43, wherein the mRNA is targeted to treat a cancer, inflammation, an infectious disease, or a genetic disease or disorder.

45. A method for the delivery of an agent into a cell comprising introducing into the cell the biomimetic proteolipid nanovesicle of any one of claims 1-22 and/or the biomimetic proteolipid nanovesicle of any one of claims 23-44.

46. The method of claim 45, wherein the agent comprises a nucleic acid.

47. The method of claim 46, wherein the agent comprises an siRNA and/or an mRNA.

48. The method of any one of claims 45-47, wherein the method is used to target delivery of the agent to inflamed tissues.

49. The method of any one of claims 45-48, wherein the method is performed in vivo, in vitro, or ex vivo.

50. The method of any one of claims 45-49, wherein the cell is human.

51. A method of treating a disease or disorder in a subject in need thereof, the method comprising administering to the subject the biomimetic proteolipid nanovesicle of any one of claims 1-22 and/or the biomimetic proteolipid nanovesicle of any one of claims 23-44.

52. The method of claim 51, wherein the agent comprises a nucleic acid.

53. The method of claim 52, wherein the agent comprises an siRNA and/or an mRNA.

54. The method of any one of claims 51-53, wherein the disease or disorder is a cancer.

55. The method of claim 54, wherein the disease or disorder is triple negative breast cancer.

56. The method of any one of claims 51-55, wherein the disease or disorder is inflammation.

57. The method of claim 56, wherein the disease or disorder is a tumor, sepsis, a traumatic brain injury, inflamed epithelia, atherosclerosis, or post traumatic osteoarthritis.
